(19) United States
(12) Patent Application Publication
(10) Pub. No.: US 2026/0161443 A1
(43) Pub. Date: Jun. 11, 2026

(54) DATA-DEPENDENCY LOGIC-DRIVEN DYNAMIC TASK MANAGEMENT SYSTEM AND METHOD

(71) Applicant: Logigle Inc., Houston, TX (US)

(72) Inventors: Kete Long, Houston, TX (US); Anuchit Anuchitanukul, Bangkok (TH); Suttipong Kanakakorn, Bangkok (TH)

(21) Appl. No.: 19/173,415

(22) Filed: Apr. 8, 2025

Related U.S. Application Data
(60) Provisional application No. 63/728,639, filed on Dec. 5, 2024.

(51) Int. Cl. G06F 9/48

(52) U.S. Cl. CPC G06F 9/485

(57) ABSTRACT

An exemplary workflow management and decision-making system and method for an automated or semi-automated decision-making platform are disclosed for a computerized/computer-guided transaction involving (i) multiple data input from a user or external data store system and (ii) associated verification operations in a workflow or work processes, e.g., loan or credit approval, claim process, and the like, that dynamically determines and tracks the verifiable data input information (e.g., financial, medical, employment, document information, or the like) to be provided by a user and verified by the decision-making platform to determine a decision outcome.

### RELATED APPLICATION

This application claims priority to, and the benefit of, U.S. Provisional Patent Application No. 63/728,639, filed Dec. 5, 2024, entitled “Method and System for Codifying Decision-Making Processes with Data-Dependency Driven Dynamic Task Management,” which is incorporated by reference herein in its entirety.

### BACKGROUND

Mobile lending app are provided by a financial institution to customers using technology having sophisticated software and automated computer-guided interfaces and processes for the customer to prepare a loan or credit application and submit supporting documentation through a smartphone, tablet app, or computer, sometimes without any in-person customer support provided by the financial institution to minimize costs and maximize process efficiency. The loan application and supporting documentation are then evaluated by an underwriter at the financial institution to evaluate and assess the risk of lending, giving credit, or providing service to the customer. Consumer loan underwriting typically includes the verification of consumer items such as employment history, salary, and financial statements; publicly available information, such as the borrower's credit history, which is detailed in a credit report; and the lender's evaluation of the borrower's credit needs and ability to pay. Examples include mortgage underwriting, auto loans, personal loans, etc. Financial institutions can streamline mobile loan applications in workflows to improve loan application processing time and cost. In some instances, financial institutions can automate some parts of the evaluation and decision-making of the loan or credit.

Mobile insurance may also be provided by an insurance company using similar technology having sophisticated software and automated computer-guided interfaces and processes for a customer to prepare and submit an insurance application (or insurance claims) through a smartphone, tablet app, or computer. Mobile insurance typically requires a customer to submit documentation to the insurance company, after which an underwriter or claim writer would evaluate and assess the risk of insuring the individual or entity. Insurance underwriters evaluate the risk and exposures of potential clients. They can decide how much coverage the client can receive, how much they should pay for it, and whether to accept the risk. Insurance claims, similarly, also require a customer to submit documentation, but a claim that is administered in accordance with the terms outlined in the insurance policy and reviewed by claim adjusters. Financial institutions (and healthcare providers, among others) can streamline mobile insurance applications or insurance claims in workflows to improve insurance application or insurance claim processing time and cost. In some instances, financial institutions or insurance companies can automate some part of the evaluation and decision-making.

Companies and governmental departments may also provide technology having sophisticated software and automated computer-guided interfaces and processes for a user to prepare and submit for services through the web. Companies and governmental departments can streamline any number of services in workflows to improve processing time and cost. In some instances, companies and governmental departments can automate some part of the evaluation and decision-making of the service. Service often also involves a user/applicant providing information and documents to the organization, and the organization gathering and evaluating the provided information to arrive at a decision point for the service (e.g., issuing a permit).

Underwriting and services provided by companies and government institutions are intentionally structured to be performed in the same and repeatable manner for deterministically, efficiency, and repeatability in the evaluation and to provide consistent results and outcomes to the applicant. The process is sometimes subject to government regulations. A well-defined work process may be structured to have (i) a short cycle time to arrive at a decision outcome, (ii) task management that is efficient, (iii) protection of user's data and privacy, and (iv) the ability to retain and recover data. Over time, though, a well-defined work process that was well structured tends to gradually become unstructured and more complex as the flow or underlying logic is modified—e.g., as new policies or new systems and tasks are added or as new connections to external workflows are implemented. To this end, over time, existing work processes can incur duplicative tasks can become inefficient (e.g., having repeated human touchpoints or suboptimal workflow), uncoordinated (unstructured logic and flow in data gathering), and inconsistent or non-replicable decision outcomes.

Managing the workflow and reconfiguring it can be complex and cumbersome and still result in untimely changes and errors, which can lead to an inability to cope or adapt to the evolving and changing nature of such decision-making processes. There is a benefit to improving the setup and technical execution of technology employed for mobile lending app, mobile insurance, and mobile-provided services of companies and government institutions.

### SUMMARY

An exemplary data-dependency logic-driven dynamic task management system and method (also referred to as a workflow management and decision-making system and method herein) are disclosed for an improved computing technology for an automated or semi-automated dynamic decision-making and task management platform for a computerized/computer-guided transaction involving (i) multiple data input from a user or external data store system and (ii) associated verification operations in a workflow or work processes, e.g., loan or credit approval, claim process, and the like, that dynamically determines and tracks verifiable data input information (e.g., financial, medical, employment, document information, or the like) to be provided by a user and verified by the decision-making platform to determine a decision outcome. Rather than a static workflow, data submitted by the user to the exemplary system and method are maintained in a data store that is dynamically evaluated with a data computation method employed in a dynamic decision-making and evaluation computing platform; the data computation method encodes the logic for the decision-making process and the dependencies of the information to define a workflow. The platform allows complex workflow and decisions with multiple possible outcomes, and outcome state and decision logic to be encoded in a combination of one or more data computation methods to which paths (i) in the logic encoded in the data computation method and (ii) corresponding to the flow of data gathering and verification processes, are determined and re-determined autonomously in a pipeline operation by a dependency resolution engine and tracker of the platform (i) at the start of the use of the logic for a set of transactions or application and during a transaction application if information is modified during the verification process and (ii) throughout later modification of the logic to later transaction and applications. The platform and its underlying operation can (i) provide a streamlined task-managed way to address changes in the customer-provided information and (ii) also reduce complexity and decision-making logic and merge that straightforward decision-making logic with automated processes that streamline the workflow for mobile loan application/process, insurance application/process, insurance claim processing, or other like services provided by companies, institutions, or government entities.

As used herein, the term “data computation method” refers to a structured data object such as a directed graph, decision tree, look-up table, set of rules, formula, and other data objects described herein, that are static for a given transaction or application (thus auditable, repeatable for a set of policies) and can be (i) later modified to incorporate new policies and new implements and (ii) later executed through the same underlying dynamically oriented process of the platform without any modification to the platform-only the logic and the underlying routing within the platform changes. The platform implements a set of software and hardware modules for a pipeline process that operates on reconfigurable decision logic encoded into a data computation method to which (i) the sequence and extent of data and document gathering and verification are determined and re-determined for a given input by the user and to which (ii) tasks and subtasks associated with the data gathering and/or verification, if needed, are determined, re-determined, and tracked in pipeline processes of the platform. The platform may be implemented with other data computation methods, including but not limited to a decision tree, directed graph, a set of rules, and a lookup table, among others described herein. The exemplary workflow management and decision-making system and method codify decision-making processes, to provide a short cycle time to arrive at decision outcomes. The automation of task generation and monitoring operates synergistically with the codified decision-making processes that reduce the programming requirement and maintenance of complex workflows and processes for banking and financial institutions and companies. Indeed, the exemplary workflow management and decision-making system and method can provide distributed and efficient task management, ease of reconfiguration, secured access protocol and client data protection, and critical data retention and recovery.

In some implementations, the exemplary system implements a module that provides logic-dependency-driven task generation based on user-provided information or documents. The tasks and documents are tracked using verification and updated flags that are maintained and managed by the system. Automatic task management and dynamic status tracking of the exemplary platform can generate multiple decision outcomes using various computation methods, including decision trees, directed graphs, tables, formulas, and rules, facilitating organizations (e.g., banks, corporations, etc.) to dynamically modify their organizational workflows and structures to achieve the best decision outcome that meets their budget requirements and client needs.

In having the decision-making logic encoded in the data computation method along with dependency for the flow of data gathering and verification processes, the platform is configured to determine and re-determine by the sequence of data gathering, tasks, and verification processes without requiring human input. In this instance, the data computation method becomes a single centralized point of modification for any new change.

The data computation method is lendable to machine learning operations that can utilize past decisions and transaction/application data to identify and/or directly generate new paths and dependencies in the logic of the data computation method or a completely new logic and dependencies. The exemplary machine-learning-powered automatic task management based on data dependencies and computations can reduce redundancy, repetitive human touchpoints, uncoordinated data gathering, and inconsistent or non-replicable decision outcomes. The exemplary machine-learning-generated data computation methods can be more efficient due to streamlined structure (e.g., pruning strategy) and more effective leading to better decision outcomes (e.g., higher predictive or explanatory power).

In some aspects, the techniques described herein relate to a system including: a task management engine configured to (i) create a template of data elements for a new transaction having a decision-making operation, (ii) create an initial task pool for the transaction, and (iii) monitor changes in a data element and data dependencies within the transaction, wherein the decision-making operation is performed via an updatable data structure including a data computation method (e.g. a decision tree, a directed graph, a set of formulas, a set of rules, or a lookup table) per the task management engine, wherein the transaction includes a container or instance that holds a set or template of data elements, wherein the data element defines a piece of information (e.g., a field, flag, value, document, intermediate result, image, biometric, drawing, etc.) that is read and updatable by a task or computed by a data computation operation that defines how data elements are derived via a set of logics contained in the updatable data structure, wherein the data computation operation implicitly creates data dependencies among data elements that are dynamically updatable for the transaction, wherein at runtime, each transaction data element and data computation method form a dynamic data dependency, wherein a change to one data element causes (i) re-computation of other data elements that depend on the respective data element and (ii) initiate or close tasks; and a task pool operatively coupled to the task management engine, wherein the task pool includes a logical container of all pending or in-progress tasks to be performed for all active transactions, wherein a new task is created and added to the task pool when the task management engine detects a data element requiring an update (e.g. input, editing, computing, verification, or a combination thereof).

In some aspects, the techniques described herein relate to a system, wherein the task management engine is configured to: analyze the transaction for incomplete information in data elements, analyze the transaction for new data elements needing verification, automatically (in a pipeline operation) create or update tasks in the task pool to respectively collect required information, assign a task that performs a direct manual operation, or assign a task that performs a computerized operation.

In some aspects, the techniques described herein relate to a system including: an interface configured to curate a user portal to receive input from a user to inputs/edits data.

In some aspects, the techniques described herein relate to a system, wherein the interface is configured to retrieve a task from the task pool and complete an operation.

In some aspects, the techniques described herein relate to a system, wherein the task management engine is configured to update, in a database, a status field of a data element to an edited status upon the data element being edited, and wherein the task management engine is configured to (i) re-compute other data elements that depend on the respective data element and (ii) initiate or close tasks.

In some aspects, the techniques described herein relate to a system, wherein the task management engine is configured to update, in a database, a status field of a data element to a verified status upon the data element being verified.

In some aspects, the techniques described herein relate to a system, wherein the updatable data structure is iteratively created, via an editor, from input from individuals in an organization servicing the transaction (e.g., institutional knowledge of senior officers or a group of individuals involved in the decision-making operation).

In some aspects, the techniques described herein relate to a system, wherein the updatable data structure is generated by a machine learning operation using training data (e.g. a collection of past transactions of an organization with realized outcomes) and existing data structure from the organization (e.g., for display within an editor for further edits).

In some aspects, the techniques described herein relate to a system, wherein the machine learning operation includes a classification and regression tree configured to empirically define cut-offs for branching or splitting of numerical data elements (e.g., for display within the editor for further edits).

In some aspects, the techniques described herein relate to a system, wherein the machine learning operation includes classification and regression tree configured to empirically define placements of data elements within the updatable data structure (e.g., for display within the editor for further edits).

In some aspects, the techniques described herein relate to a system, wherein the machine learning operation includes classification and regression tree configured to determine new branches responsive to new data received in relation to the training data (e.g., continual learning) (e.g., for display within the editor for further edits).

In some aspects, the techniques described herein relate to a system, wherein the machine learning operation includes classification and regression tree configured to prune unnecessary branches within the updatable data structure (e.g., to produce more efficient decision-making processes) (e.g., for display within the editor for further edits).

In some aspects, the techniques described herein relate to a system, wherein the transaction is directed to a loan or insurance application, and wherein the decision operation is directed to an approval, declination, or classification of the loan or insurance application within a pipeline operation of the task management engine (e.g., with no or minimal loan officer or underwriter input).

In some aspects, the techniques described herein relate to a system, wherein the transaction is directed to a building permit application, and wherein the decision operation is directed to an approval, declination, or request for more additional for the building permit application using a pipeline operation of the task management engine (e.g., with no or minimal county or town officer or clerk input).

In some aspects, the techniques described herein relate to a system, wherein the transaction is directed to a patient check-in process for a clinic, hospital, or healthcare service facility, and wherein the decision operation is directed to a routing operation for the patient check-in process using a pipeline operation of the task management engine (e.g., with no or minimal nurse or receptionist input).

In some aspects, the techniques described herein relate to a system, wherein the transaction is directed to a collateral of a collateralized financial product, wherein the decision operation is directed to validation or maintenance of the collateralized financial product using a pipeline operation of the task management engine (e.g., with no or minimal collateral manager input).

In some aspects, the techniques described herein relate to a system, wherein the transaction is directed to an insurance claim application, and wherein the decision operation is directed to an approval, declination, or request for additional information for the insurance claim application using a pipeline operation adjuster input).

In some aspects, the techniques described herein relate to a system, wherein the formula in the updatable data structure is manually created.

In some aspects, the techniques described herein relate to a system, wherein the formula is embedded in a data computation method (e.g., a decision tree, a directed graph, a lookup table, etc.)

In some aspects, the techniques described herein relate to a system, wherein the formula is implemented outside of a data computation method (e.g., a decision tree, a directed graph, and a lookup table, etc.)

In some aspects, the techniques described herein relate to a system, wherein the rule in the updatable data structure is manually created.

In some aspects, the techniques described herein relate to a system, wherein the rule is embedded in a data computation method (e.g., a decision tree, a directed graph, and a lookup table, etc.)

In some aspects, the techniques described herein relate to a system, wherein the rule is implemented outside of a data computation method (e.g., a decision tree, a directed graph, and a lookup table, etc.)

In some aspects, the techniques described herein relate to a system, wherein decision outcomes (end nodes, or leaf nodes) in the data computation methods are defined by one or multiple data elements.

In some aspects, the techniques described herein relate to a system, wherein the data computation method is a directed acyclic graph.

In some aspects, the techniques described herein relate to a system, wherein the data computation method is a directed cyclic graph.

In some aspects, the techniques described herein relate to a system, wherein the data computation method is a weighted directed graph.

In some aspects, the techniques described herein relate to a system further including a path analyzer analyzing all possible paths from a decision outcome (end node, or leaf node) to root node.

In some aspects, the techniques described herein relate to a system, wherein the path analyzer identifies and evaluates multiple paths for efficiency, priority, and conflict resolution.

In some aspects, the techniques described herein relate to a system, wherein the path analyzer identifies and evaluates a loop in a path.

In some aspects, the techniques described herein relate to a computerized method including: providing a task management engine configured to (i) create a template of data elements for a new transaction a decision-making operation, (ii) create an initial task pool for the transaction, and (iii) monitor changes in a data element and data dependencies within the transaction; analyzing the transaction for incomplete information in data elements; analyzing the transaction for new data elements needing verification; automatically (in a pipeline operation) creating or updating tasks in the task pool to respectively collect required information; assigning a task that performs a direct manual operation; and assigning a task that performs a computerized operation.

In some aspects, the techniques described herein relate to a non-transitory computer-readable medium having instructions stored thereon, wherein execution by a processor causes the processor to: analyzing a transaction for incomplete information in data elements; analyzing the transaction for new data elements needing verification; automatically (in a pipeline operation) creating or updating tasks in a task pool to respectively collect required information; assigning a task that performs a direct manual operation; and assigning a task that performs a computerized operation.

### BRIEF DESCRIPTION OF DRAWINGS

FIGS. 1A-1D shows an exemplary workflow management system and method and associated engines, operations, and elements. FIGS. 1A and 1B each shows an exemplary workflow management and decision-making system and method for an automated or semi-automated decision-making platform for a computerized/computer-guided transaction involving (i) multiple data input from a user or external data store system and (ii) associated verification operations in a workflow or work processes that can dynamically determine and track the verifiable data input information to be provided by a user and verified by the decision-making platform to determine a decision outcome. FIG. 1C shows an example data element of the exemplary system. FIG. 1D shows an example high-level user-perspective diagram when a user interacts with the exemplary system.

FIGS. 2A-2B show an example operation flow of the task management engine and how data computation methods of various types interact in a task management operation. FIG. 2A shows an example operation flow of the task management engine, which can comprise 6 steps.

FIG. 2B shows example interactions between data computation methods of various types in the task management operation.

FIGS. 3A-3I shows example data computation methods as various types of structures, including algebraic equations, formulas in a programming language, tree structure, multilayered decision tree, decision-directed graph, decision-directed graph with a loop, and load decision table. FIG. 3A shows example data computation methods. FIG. 3B shows an example computation of data elements using algebraic equations and formulas in programming language style. FIG. 3C shows an example computation of data elements using a tree structure. FIG. 3D shows an example computation of data elements using a multilayered decision tree. FIG. 3E shows an example computation of data elements in illness treatment. FIG. 3F shows an example data computation method as a decision tree. FIG. 3G shows an example data computation method as a decision-directed graph. FIG. 3H shows an example data computation method as a loan decision-directed graph with a loop. FIG. 3I shows an example data computation method as a loan decision table.

FIGS. 4A-4I shows example data computation methods as various types of trees and graphs. FIG. 4A shows an example diagram showing various embodiments of a data computation method, including graphs, directed graphs, directed acyclic graphs, and decision trees. FIG. 4B shows an example directed graph configuration of a computation method. FIG. 4C shows an example decision tree configuration for the data computation method. FIG. 4D shows an example directed acyclic graph (DAG) configuration for the data computation method. FIG. 4E shows an example directed cyclic graph (DCG) configuration for the data computation method. FIG. 4F shows an example DCG having multiple paths between an end node L₁ and the root node No, wherein there is a path without a loop. FIG. 4G shows an example multi-path analysis method #1. FIG. 4H shows an example multi-path analysis method #2. FIG. 4I shows example multi-root-node graphs having multiple paths between multiple root nodes and leaf nodes.

FIGS. 5A-5I show example tasks and data dependencies in conventional workflows and workflows configured by the exemplary system. FIG. 5A shows an example task defined as an input screen (e.g., for data entry), an editing prompt, or an action prompt. FIG. 5B shows an example task requiring data to create data dependency relationships between data elements. FIG. 5C shows an example of conventional workflow and workflow configured by the exemplary system. FIG. 5D shows example time stamps recorded in a conventional workflow and a workflow configured by the exemplary system. FIG. 5E shows an example of multi-user and multi-channel configurations in a conventional workflow and a workflow configured by the exemplary system. FIG. 5F shows an example change to a conventional workflow and workflow configured by the exemplary system. FIG. 5G shows an example of dynamic dependency between a data computation method and a transaction. FIG. 5H shows an example data dependency configured to be dynamic for each transaction. FIG. 5I shows the cascading status of data elements via data dependency in the exemplary system.

FIGS. 6A-6F show an example machine learning (ML) processing tool (i.e., ML module) used in the exemplary system and example trees and graphs generated by the ML processing tool. FIG. 6A shows an example ML processing tool used in the exemplary system to generate a decision tree. FIG. 6B shows an example decision tree generated by the conventional generation (i.e., heuristic) method. FIG. 6C shows an example decision tree generated by the intelligent generation method employed by the exemplary system. FIG. 6D shows an example ML processing tool used in the exemplary system to generate a directed graph. FIG. 6E shows an example AI training system for the ML processing tool using a Classification and Regression Tree (CART). FIG. 6F shows an example directed graph generated by an ML processing tool employed by the exemplary system.

FIGS. 7A-7B show various applications for the exemplary system. FIG. 7A shows the directed graph representing a medical diagnosis.

FIG. 7B, subpanels (a)-(c) each shows the table lookup applications for the exemplary system.

### DETAILED DESCRIPTION

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. For example, [1] refers to the first reference in the list. All references cited and discussed in this specification are incorporated herein by reference in their entirety and to the same extent as if each reference was individually incorporated by reference.

### Example System

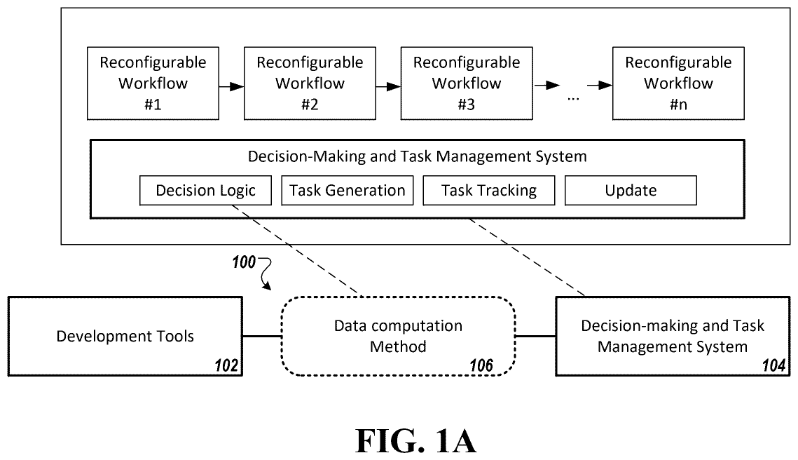

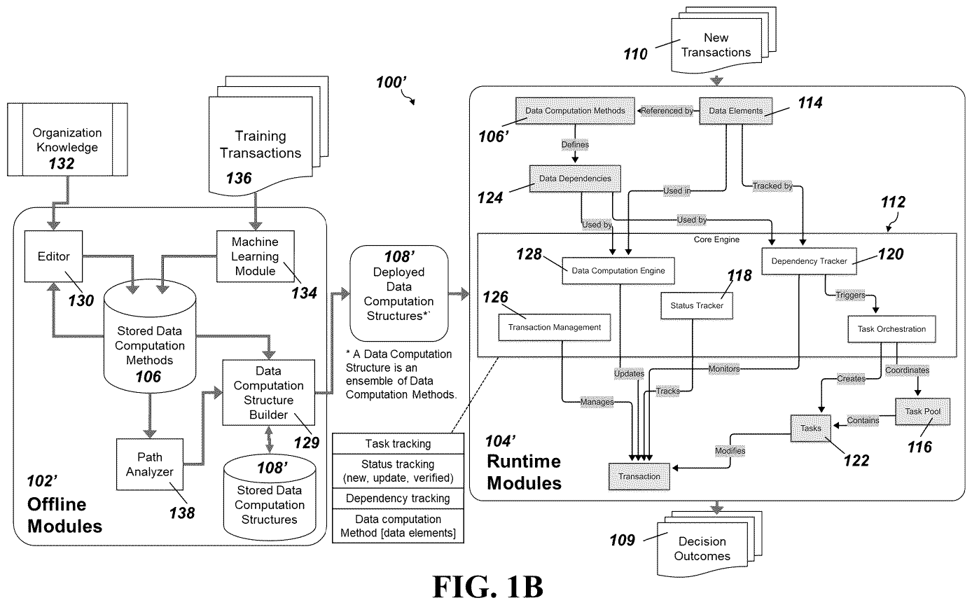

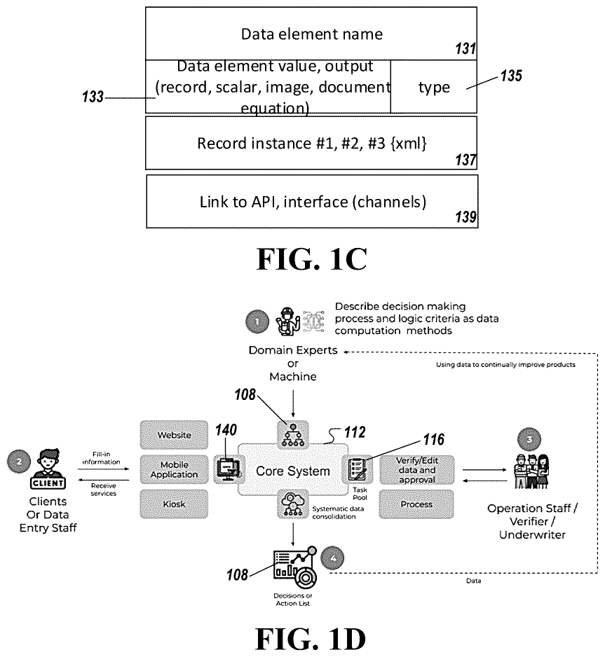

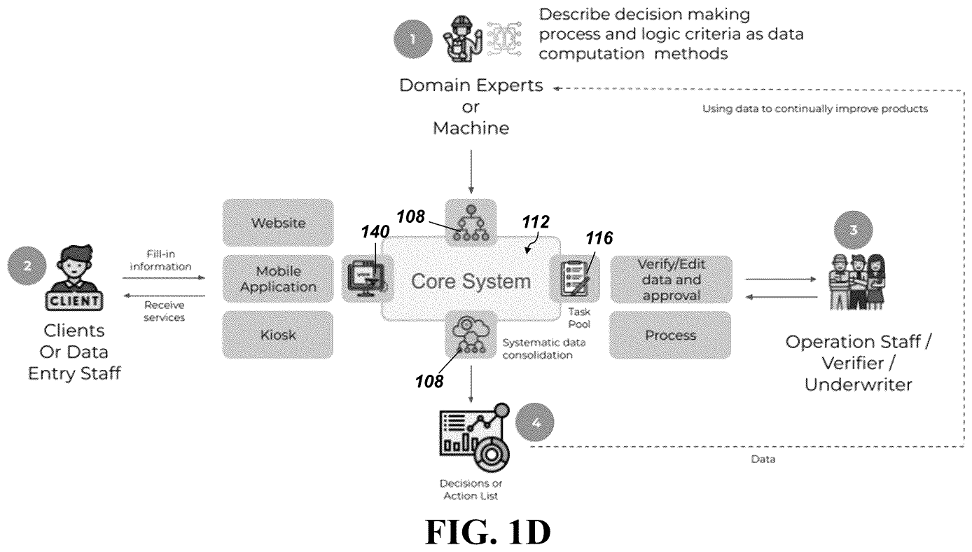

FIGS. 1A and 1B show an exemplary workflow management and decision-making system and method (also referred to as a decision-making and task management system herein) for an automated or semi-automated dynamic decision-making platform for a computerized/computer-guided transaction involving (i) multiple data input from a user or external data store system and (ii) associated verification operations in a workflow or work processes that can dynamically determine and track the verifiable data input information to be provided by a user and verified by the decision-making platform to determine a decision outcome.

In the example shown in FIG. 1A, the workflow management and decision-making system 100 and method comprises a development module 102 (shown as “development tools” 102) that generates data computation method 106 for a decision-making and task management system 104. The decision-making and task management system 104 includes a plurality of software modules that include dynamically generated and updatable decision logic that is defined by a data computation method 106 generated by the development tool 102. The decision-making and task management system 104 additionally includes task generation and task tracking modules that operate synergistically with the codified decision-making processes per the data computation method 106 to generate tasks to initiate, track, and analyze documentation/information provided by the user. The dual and dynamic task-associated generation/tracking features and logic editing features allow the workflow management and decision-making system 100 and method to automate and reduce the programming and reprogramming (maintenance) requirement when changing/updating the decision-making process of complex workflows and processes for banking and financial institutions and companies.

The decision-making and task management system 104 and development tools 102 are configured to maintain independent continuous operation with one another. To this end, data computation method 106 and its underlying logic, when created by the development tools 102 is still linked to push updates to the data computation method 106 made at the development tools 102 to the decision-making and task management system 104 (analogous to a compiler and runtime system). The decision-making and task management system 104 includes functional modules that can take a data computation method 106 or an updated version of the same and automatically generate and re-generate tasks and workflows using data dependencies defined in the data computation method 106. To this end, updates can be made to the underlying decision process, new or revised requirements for documentation and information, etc., at the development tools 102 that are then automatically implemented, reimplemented, and monitored as an optimized workflow by the decision-making and task management system 104.

Each transaction 110 is instantiated through a data computation method 106 (developed by the development tools 102) as a container/instance and holds a set of data elements and is then dynamically tracked by the task management engine for status/changes in the data elements and data dependencies. Data elements may be designated a status of new/default, edited, or verified. New and edited status can initiate tasks for a data element. Verified status indicates the data element is completed with information/documentation, and the information/documentation is verified. Statuses may cascade to downstream elements that depend on it. Different statuses may be tracked by the status tracker.

Each data element of the data computation method represents individual pieces of information (e.g. data, fields, flags, documents, images, intermediate results, etc.). Each data element can be read or updated by a task or computed by the task management engine in accordance with a data computation method. A task may be performed by an associated agent, e.g. underwriter, credit officer, customer, computer module, etc.

Data computation methods—as a set of logics-define how certain data elements are derived from others (e.g., formulas, decision trees, table lookups, directed graphs, a set of rules). Data computation methods implicitly create data dependencies among data elements by a set of logics governing a given data computation method. Graphs as a data computation method have graph structures that show the flow of information; data elements thus have implicit definitions according to their location and sequencing in the graph. Look-up tables have explicit structure operation according to data as placed in the structure; data elements of the look-up table thus have implicit definitions according to their location in the table. Common to each of the data computation methods, because the relationships can change with different values, these dependencies are dynamically defined and thus dynamic within each transaction.

In addition, the decision-making and task management system 104 employs (i) decision logic of the data computation method 106 to make decisions for a given transaction (e.g., approve for X, approve for Y, disapprove, etc.) using user-provided information or documentation instantiated as data elements in the data computation method 106 and (ii) task management features to track changes and verified states of customer provided information and documentation. The decision-making and task management system 104 thus provides a system workflow that addresses updates and changes to information provided by customers with the need to redo an application or involve a person (e.g., bank employee) in the process. In FIG. 1A, a number of reconfigurable workflows (shown as reconfigurable workflow #1, #2, #3, . . . #n) are shown that can correspond to different documentation and information aggregation operations performed for a mobile loan application, a mobile insurance application, a mobile-provided service application.

FIG. 1B shows an example implementation of the development tools 102 (shown as 102′) and decision-making and task management system 104 (shown as 104′). In the example shown in FIG. 1B, the workflow management and decision-making system 100′ is configured with (i) development tools 102 (shown as “Offline Modules” 102′) and (ii) decision-making and task management system 104 (shown as “Runtime Modules” 104′, where the runtime modules 104′ receive a data computation structure 108 (shown as 108′) corresponding to a plurality of data computation methods 106 (shown as 106′) developed in the offline modules 102′. The offline modules 102′ provide user-developed data computation methods 106 (manual or automated, e.g., via machine learning) as a data computation structure 108 to the runtime modules 104′, which initialize an instance of the data computation structure 108 for each transaction to be handled by the decision-making and task management system 104′. Data computation method 106 is the logic and logic flow for the data element as recorded, e.g., by an editor of the offline module 102′. Data computation methods 106 can also be generated using machine learning algorithms that are editable in the editor and can be imported for use in the generating of the data computation structure. The data computation structure is an ensemble derived from the data computation method.

In FIG. 1B, the runtime modules 104′ are configured to generate decision outcomes 109 for each new transaction 110 using a task management engine 112 (shown as core engine). The offline modules 102′, coupled with the runtime modules 104′, are configured to generate, store, and transmit data computation methods in the data computation structure 108 to the runtime modules 104′. The runtime modules 104′ generate a template application to which individual instances of the template application can be invoked for a new mobile loan application, mobile insurance application, mobile-provided service application, or the like. Indeed, the specific application is generated by or for a particular institution or company for a given service.

Task management engine. In the example shown in FIG. 1B, the task management engine 112 (shown as core system 112) is configured to (i) track data elements 114 of a data computation method 106 for a new transaction 110, (ii) create a task pool 116 for the transaction 110, and (iii) monitor for status changes in the data element via a status tracker 118 and data dependencies via a dependency tracker 120 within a transaction 110, where the transaction 110 can comprise a container or instance that holds the data element 114. The task management engine 112 is configured to initialize the data elements 114 for a data computation method 106′ received from the development system 102; the engine 112 services the data element 114 and tasks 122. Data elements 114 are instances of the user's information or documentation that are received and stored in the decision-making and task management system 104. As users fill in their information or provide documentation, the data is stored as a data element.

The decision-making operation is performed via the data computation structure 108 comprising a data computation method 106′ (e.g., a decision tree, a directed graph, a set of formulas, a set of rules, or a lookup table) per the task management engine 112. The data element can define a piece of information (e.g., fields, flags, documents, images, intermediate results, etc.) that is readable and updatable by a task 122 or computed by a data computation operation that can define how data elements are derived via a set of rules contained in the updatable data structure 108. The data computation operation can implicitly create data dependencies among data elements that are dynamically updatable for the transaction 110. At runtime, each transaction data element 114 and data computation method 106′ form a dynamic data dependency. A change to one data element can cause (i) re-computation of other data elements that depend on the respective data element and (ii) initiate or close tasks.

The task management engine 112 can be configured to (i) analyze the transaction 110 for incomplete information in data elements 114, (ii) analyze the transaction for new data elements needing verification (via data computation engine 128 or transaction management module 126), (iii) automatically (e.g., in a pipeline operation) create or update tasks 112 in the task pool 116 to respectively collect required information, (iv) assign a task (e.g., 112) that performs a direct manual operation, or (v) assign a task 112 that performs a computerized operation.

The task management engine 112 can update, in a database, a status field of a data element to an edited status upon the data element being edited and can also (i) re-compute another data element that depends on the data being edited and (ii) initiate or close tasks. The task management engine 112 can also update, in the database, the status field of a data element to a verified status upon the data element being verified.

Indeed, rather than a static workflow, data submitted by the user are maintained as data elements (e.g., in a data computation structure 108) that are linked to a data computation method 106′, which encodes the logic for the decision-making process and the underlying workflow. Complex workflow and decisions with multiple possible outcomes and outcome states may be encoded a data computation method 106′ to which paths (i) in the logic encoded in the data computation method and (ii) corresponding to the flow of data gathering and verification processes, are determined and re-determined by a dependency resolution engine and tracker 120 of the development platform 102 and runtime platform 104 (i) at the start of the use of the logic for a set of transactions or application and (ii) throughout later modification of the logic to later transaction and applications. To this end, the logic, while static for a given transaction or application (thus auditable, repeatable for a set of policies), can be (i) later modified to incorporate new policies and new implements and (ii) later executed through the same underlying dynamically oriented process of the platform without any modification to the platform—only the logic and the underlying routing within the platform changes.

Task pool. The task pool 116 is coupled to the task management engine 112, wherein the task pool 116 can comprise a logical container of all pending or in-progress tasks 122 to be performed for all active transactions. Each new task 122 can be created and added to the task pool 116 when the task management engine 112 detects a data element 114 requiring an update (e.g., input, editing, verification, or a combination thereof).

Tasks. A task 122 is a basic unit of human or automated work in the task pool 116, including data entry, editing, validation, approval, or specialized verification task. A task 122 can (i) prompt a user to input/modify data, (ii) trigger a validation or an authorization step (e.g., supervisor override), or (iii) mark a data element as verified. Task execution can change the values or statuses of one or more data elements, known as cascading updates in the dependent elements.

Interface. In some embodiments, the exemplary system can comprise an interface (shown as 140 in FIG. 1C) configured to curate a user portal (e.g., via website, mobile applications) to receive input from a user to input/edit data. The interface is also configured to retrieve a task 122 from the task pool 116 and complete an operation.

Updatable data structure. The updatable data structure (shown as data computation structure 108, 108′) can be iteratively created, via an editor 130, from input from individuals in an organization servicing the transactions (e.g., organization knowledge 132 of senior officers or a group of individuals involved in the decision-making operation). The updatable data structure 108 can also be generated for a new type of transaction by a machine learning (ML) module 134 (i.e., ML processing tool) using training data 136 (e.g., a collection of past transactions of an organization with realized outcomes) and existing data structure from the organization (e.g., for display within an editor for further edits).

In the updatable data structure 108, the formula can be (i) manually created, (ii) embedded in a data computation method (e.g. a decision tree, a directed graph, a lookup table, etc.), (iii) implemented outside of a data computation method 106′ (e.g. a decision tree, a directed graph, and a lookup table, etc.). The formulas are standalone as its own data computation method, not only embedded in a decision tree.

In the updatable data structure 108, the rule can be (i) manually created, (ii) embedded in a data computation method (e.g. a decision tree, a directed graph, a lookup table, etc.), or (iii) implemented outside of a data computation method (e.g. a decision tree, a directed graph, and a lookup table, etc.).

Data element. FIG. 1C shows an example data element of the exemplary system. In FIG. 1C, the data element of the structure may have a name 131, a data payload 133, a definition type 135, records 137, and a link to an external source 139. Payload 133 includes the information to be used in the logic. Records 137 are additional information that may be associated with the payload information 133. Data elements can include information to trigger tasks, e.g., an executable line command, for example, for an API interface for an external system or to a module in the platform.

Editor. The editor module (shown as 130 in FIG. 1B) employed by the offline modules 102′ provides a user interface to allow the data computation method and its associated rules to be generated or modified. The editor may allow data elements to be added. Properties of the data elements (e.g., as shown in FIG. 1C) may be defined by the editor 130.

In some embodiments, editor 130 can import a data object (e.g., a directed graph) from a file.

Machine learning operation. The machine learning operation (shown as 134 in FIG. 1B), employed by the offline modules (shown as 102′ in FIG. 1B), can comprise a classification and regression tree configured to (i) define cut-offs for branching or splitting of numerical data elements, (ii) define placements of data elements within the updatable data structure, (iii) determine new branches responsive to new data received in relation to the training data (e.g., continual learning), or (iv) prune unnecessary branches within the updatable data structure (e.g., to produce more efficient decision-making processes) (e.g., for display within the editor for further edits).

The offline modules 102′ can further comprise a path analyzer (shown as 138 in FIG. 1B) configured to (i) analyze all possible paths from a decision outcome (i.e., end node) to a root node, (ii) identify and evaluate multiple paths for efficiency, priority, and conflict resolution, and (iii) identify and evaluate a loop in a path.

The transactions performed by the exemplary system in FIG. 1B can be directed to a loan or insurance application, a building permit application, a research grant proposal, a patient check-in workflow for a clinic or hospital, a collateralized financial product, or an insurance claim application, wherein the decision operation (shown as a decision or action list) can be directed to an approval, declination, or request for additional information.

Data elements can be central as tasks, and computation methods either update or derive their values. Task management (by task management engine 112) can be dynamic as there is no rigid, predefined workflow; tasks can emerge and disappear in response to data changes and dependencies. Verification tasks can explicitly model approval or authorization steps by changing a data element's status from “edited” to “verified”. Incremental changes in task management can propagate automatically due to the dependency-aware design of the exemplary system, as there is no need to restart the entire workflow/process when there is a change.

FIG. 1D shows an example high-level user-perspective diagram when a user interacts with the exemplary system.

### Example Method

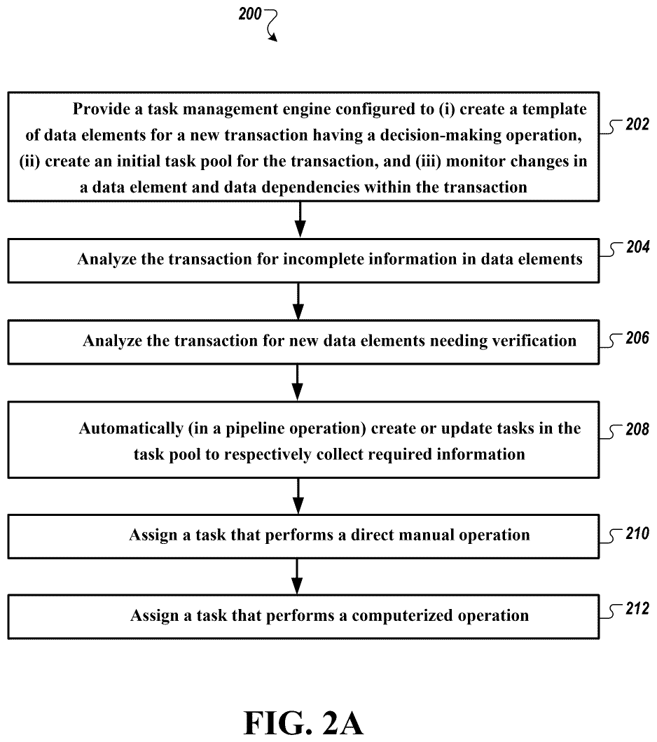

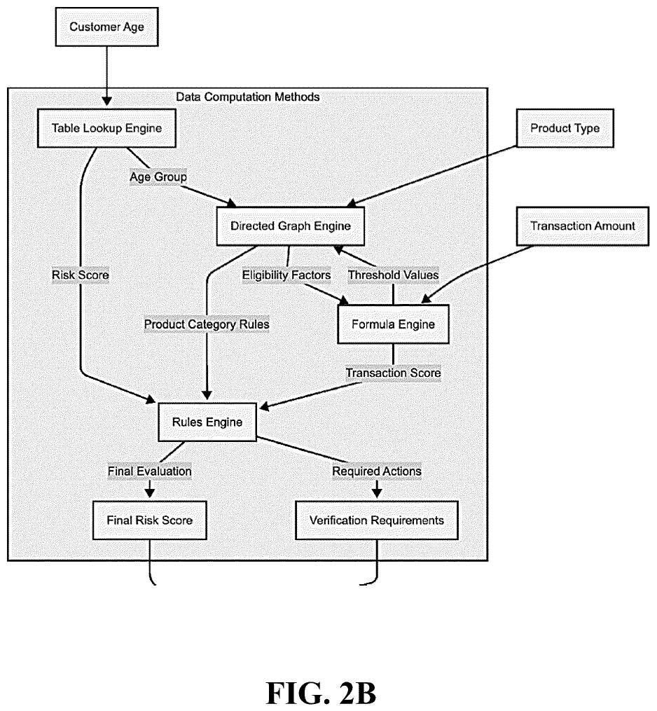

Example method #1. FIG. 2A shows an example operation flow 200 of the task management engine, which can comprise 6 steps. At step 202, the task management engine can provide a task management engine configured to (i) create a template of data elements for a new transaction having a decision-making operation, (ii) create an initial task pool for the transaction, and (iii) monitor changes in a data element and data dependencies within the transaction.′

At step 204, the task management engine can analyze the transaction for incomplete information in data elements. At step 206, the task management engine can analyze the transaction for new data elements needing verification. At step 208, the task management engine can automatically (in a pipeline operation) create or update tasks in the task pool to respectively collect required information. At step 210, the task management engine can assign a task that performs a direct manual operation. At step 212, the task management engine can assign a task that performs a computerized operation.

Example method #2. The task management operation starts when a new transaction is created with a template of data elements (all starting in “new/default” status). The data computation methods then define how some data elements depend on others. Tasks requiring certain prerequisite data elements are also associated, creating additional dependencies. Templates can be generated from a more complex data computation structure, not just one decision tree.

The task management engine then analyzes which data elements are missing or need verification and automatically creates or updates tasks in the task pool to collect required information or to perform verification steps. A user (or external system) then retrieves the task from the task pool, inputs/edits data, or completes a verification or validation step, which updates the relevant data elements in the transaction.

Once a data element is edited, its status changes to “edited”, and anything that depends on it may be re-computed or re-verified by the task management engine. If a “verification task” is completed, the data element can become “verified” (assuming its prerequisites are also verified or default).

After a data element changes, any data computation methods referencing that element are re-evaluated, updating other dependent data elements and potentially triggering new tasks (e.g., if a newly computed value also requires verification). Some tasks may be automatically marked complete once they are no longer relevant.

The task management engine continues until the overall transaction objectives are met (e.g., final decision outcome). The final “verified” status of key data elements indicates that all required verifications and authorizations have been completed.

### Example Data Computation Method

FIG. 2B shows example interactions between data computation methods of various types in the task management operation.

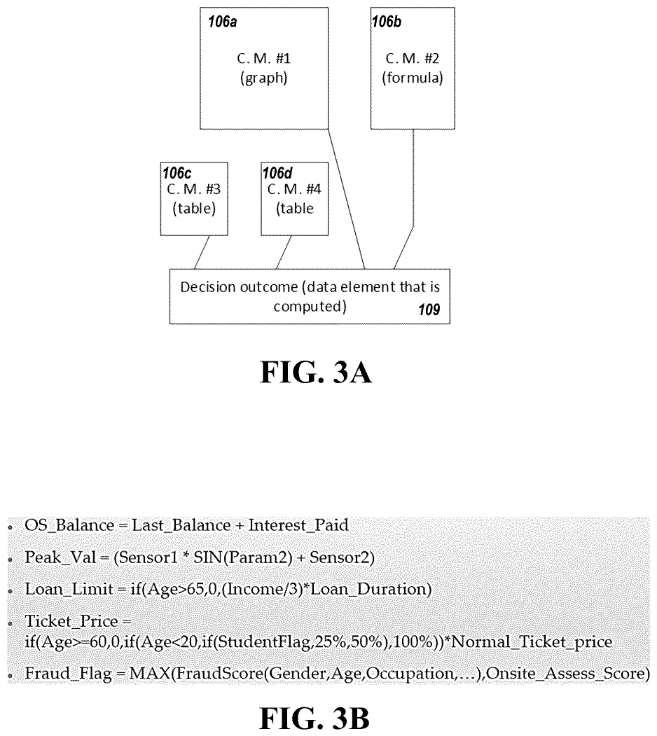

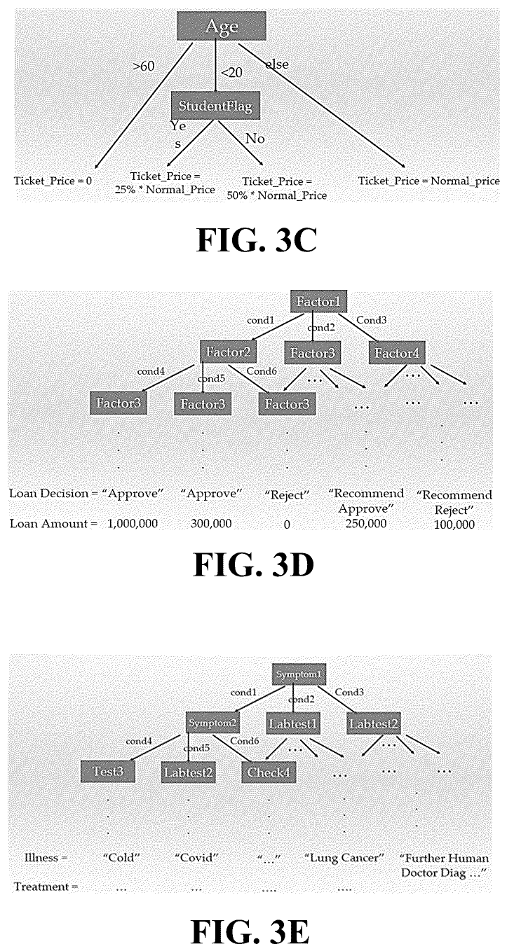

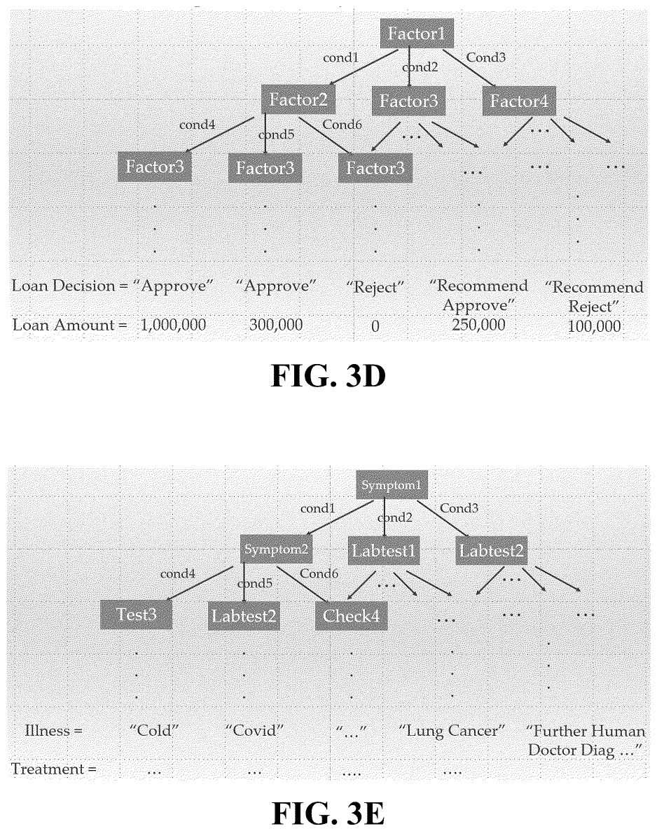

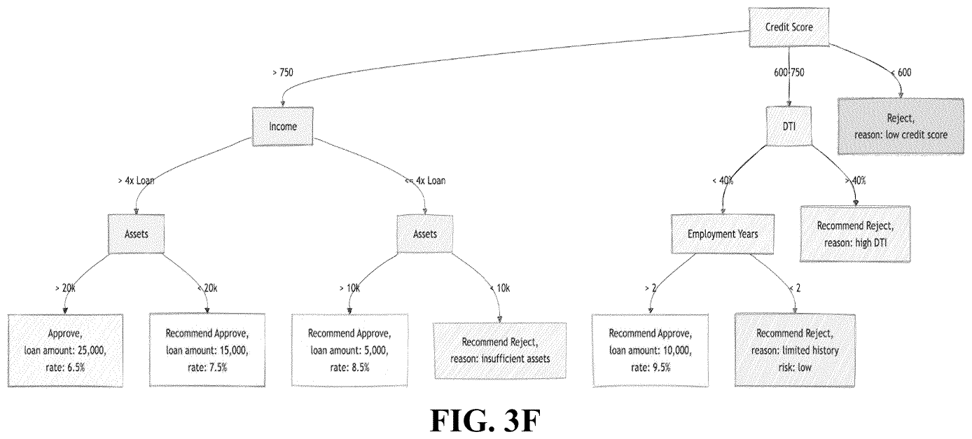

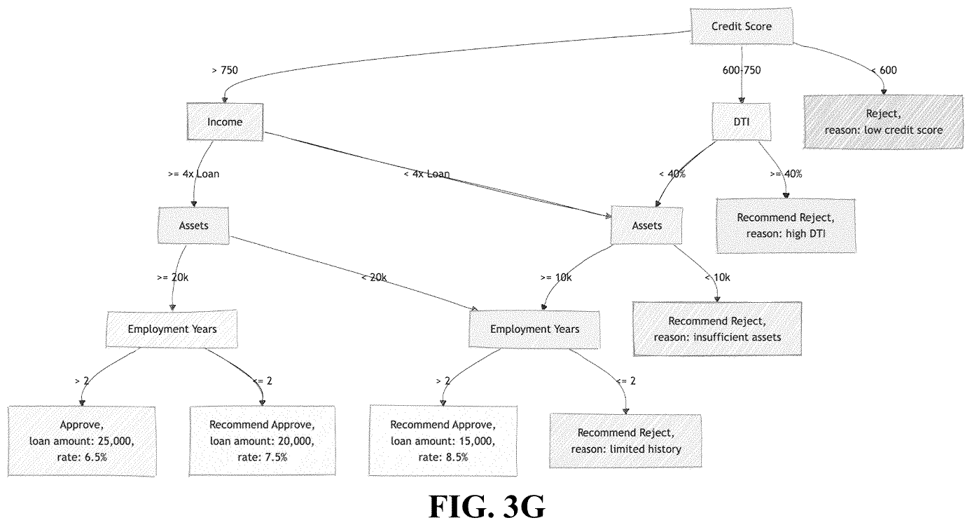

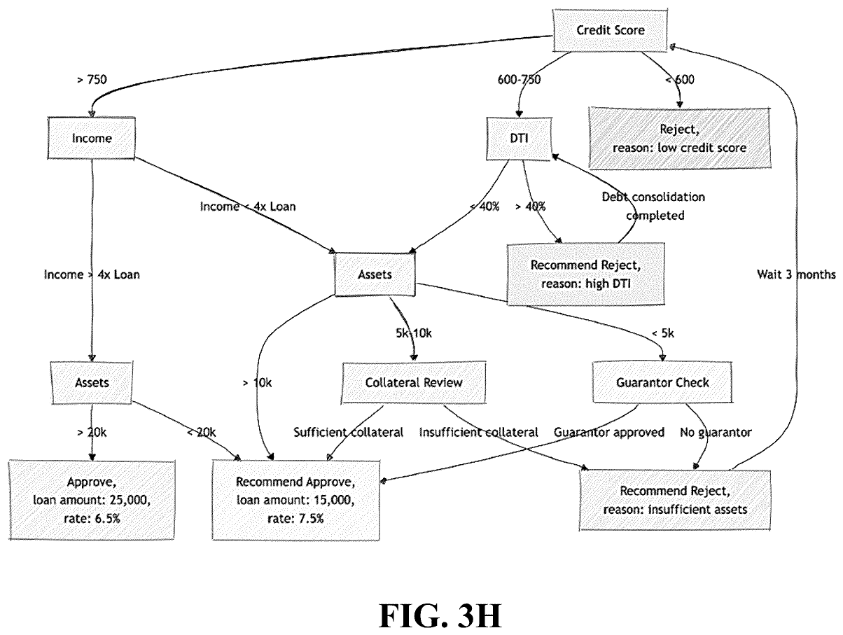

FIG. 3A shows example data computation methods 106a-106d. In FIG. 3A, the data computation methods 106a-106d (shown as C.M. #1-C.M. #4) each encodes the logic for the decision-making process and the underlying workflow in a data computation structure. Complex workflow and decisions with multiple possible outcomes and outcome states may be encoded as a directed graph, decision tree, look-up table, and other data object described herein—as a data computation method to which paths (i) in the logic encoded in the data computation method and (ii) corresponding to the flow of data gathering and verification processes, are determined and re-determined by the platform. The data computation structure can be a collection of data computation methods, i.e., decision tree, directed graph, table, and standalone formula, as shown below.

Data computation methods (e.g., 106a-106d) in the structure may produce conflicting/different decision outcomes 109. Multiple values are not allowed in a decision outcome data element. A “fusion” module can take intermediate decision outcomes from multiple data computation methods and generate a more robust single decision outcome. Examples of the operation of the fusion module may be based on the most conservative outcome, majority vote wins, overrides, decision outcome grid, etc.

In some embodiments, data computation methods are organized in sequential order; that is, an intermediate decision outcome can be defined as an input to the next data computation method. The organization order denotes that later-defined methods dominate the previous ones.

Example Data Computation Method #1. Computation of data elements can be defined as an algebraic equation, an assignment (in programming language style), a function, or a formula containing the references to the value of other data elements. FIG. 3B shows an example computation of data elements using algebraic equations and formulas in programming language style.

In FIG. 3B, the computation method can define data dependency among data elements, not only in a static relationship but in a dynamic way, as some dependency may change as the value of some data element changes.

Discrete structures such as graphs, trees, and tables can be used to define data computation methods for one or many data elements altogether. FIG. 3C shows an example computation of data elements using a tree structure.

Structures such as multilayered decision trees with thousands of nodes and simultaneous computation of many data elements can also define data computation methods. FIG. 3D shows an example computation of data elements using a multilayered decision tree.

Besides financial transactions, the data computation methods can also be defined in many subject areas. FIG. 3E shows an example computation of data elements in illness treatment.

Example Data Computation Method #2. Data computation methods can also be defined using (i) directed graphs with nodes (i.e., vertices) and edges or (ii) tables.

In the exemplary system, each data computation method can produce multiple outputs from data elements (e.g., fields). For example, a loan approval-directed graph may generate results per Table 1.

TABLE 1
1. approval=“recommend approve”, risk_score=2
2. approval=“auto approve”, loan_amount=500
3. risk_score=“high”, max_loan_amount=100
4. approval=“recommend approve”, loan_amount=2000, minimum_monthly_payment=500
5. approval=“recommend reject”

The data computation method shown in Table 1 can be demonstrated as a decision tree. FIG. 3F shows an example data computation method as a decision tree. In FIG. 3F, besides the approval result, leaf nodes can contain additional outputs such as interest rate, decision reason, and risk level.

The data computation method shown in Table 1 can also be demonstrated as a decision-directed graph. FIG. 3G shows an example data computation method as a decision-directed graph. In FIG. 3G, besides the approval status, leaf nodes can contain additional outputs such as interest rate, decision reason, and loan amount.

FIG. 3H shows an example data computation method as a loan decision-directed graph with a loop.

FIG. 3I shows an example data computation method as a loan decision table.

Different computation methods may produce overlapping results. For example, both the loan decision table and the loan approval directed graph (in FIGS. 3F-3I) can provide outputs for approval status and loan amount. Users of the exemplary system can choose which computation method's results to use. A fusion model may be utilized for such a situation.

The computation methods may produce different types of data elements as outputs. For instance, in a loan application system, outputs such as approval status, loan amount, interest rate, and decision reason may come from a directed graph. However, the risk score can be calculated separately using a formula defined per Equation 1.

$$Risk Score = f ( assets , creditScore , DTI ) \qquad ( Eq . 1 )$$

In Equation 1, risk=w₁*creditScore+w₂*assets−w₃*DTI, wherein the weights w₁=0.4 for creditScore, w₂=0.3 for assets, w₃=0.3 for DTI; and normalized inputs creditScore ∈[300-850], assets ∈[0-100 k], and DTI ∈[0-100%].

### Example Graphs as Data Computation Methods

In some embodiments, data computation methods can be implemented as directed graphs with nodes (i.e., vertices) and edges.

Non-directed (undirected) graph. A non-directed graph has edges with no direction, wherein information can flow freely between connected vertices in both directions.

Directed graph. A directed graph has edges with a specific direction, wherein information can only travel from one vertex to another in a particular way. A directed graph represents a one-way relationship, whereas a non-directed graph represents a two-way relationship.

Table 2 shows definitions of various types of directed graphs.

TABLE 2

| Types of directed graphs | Description |
|---|---|
| Directed cyclic graph (DCG) | A directed graph with a directed cycle |
| Directed Acyclic Graph | A directed graph with no directed cycles. DAG consists of |
| (DAG) | nodes (i.e., vertices) and edges (i.e., arcs), with each edge |
| | directed from one node to another, such that following those |
| | directions can never form a closed loop. |
| Decision Tree: | A decision tree is a type of directed acyclic graph (DAG) |
| | where each node has only one incoming edge, meaning it |
| | follows a strict hierarchical structure, while a DAG can have |
| | more complex relationships between nodes with multiple |
| | incoming edges, allowing for more flexible connections |
| | between different parts of the graph. |

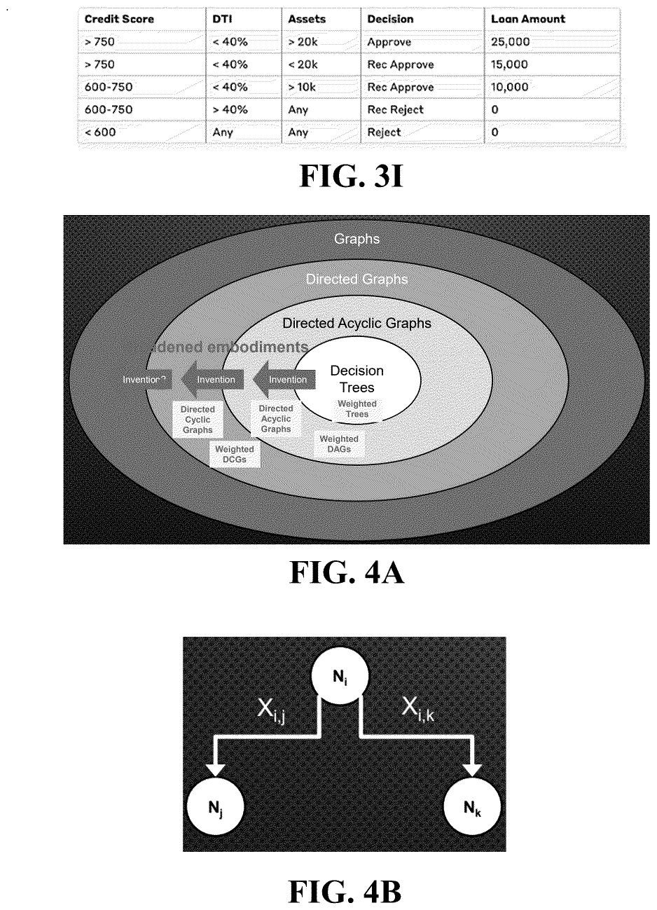

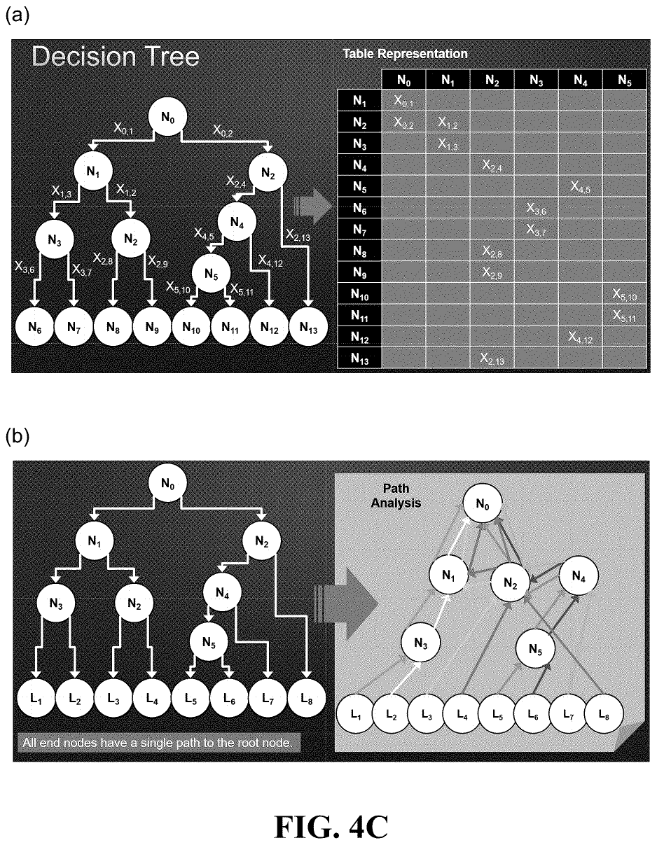

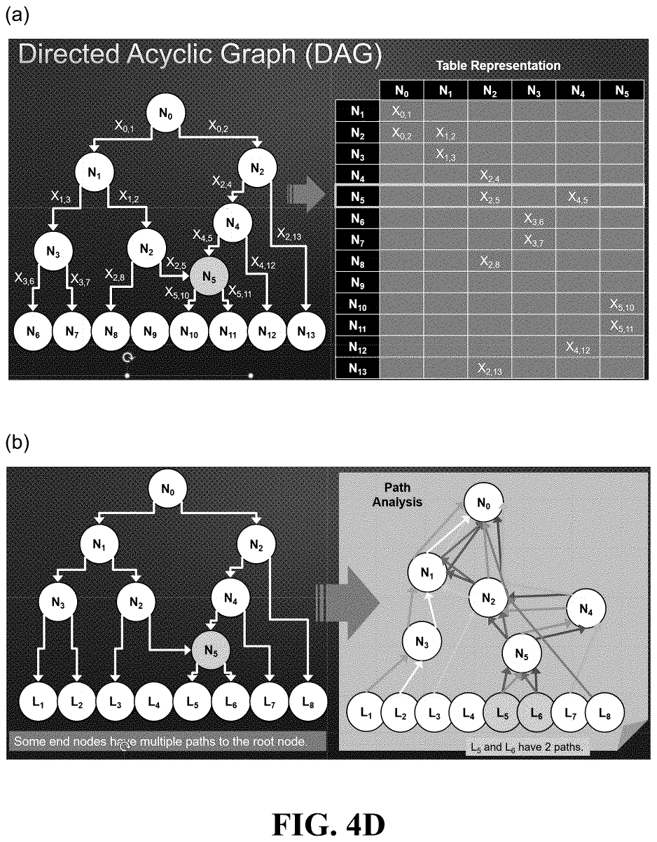

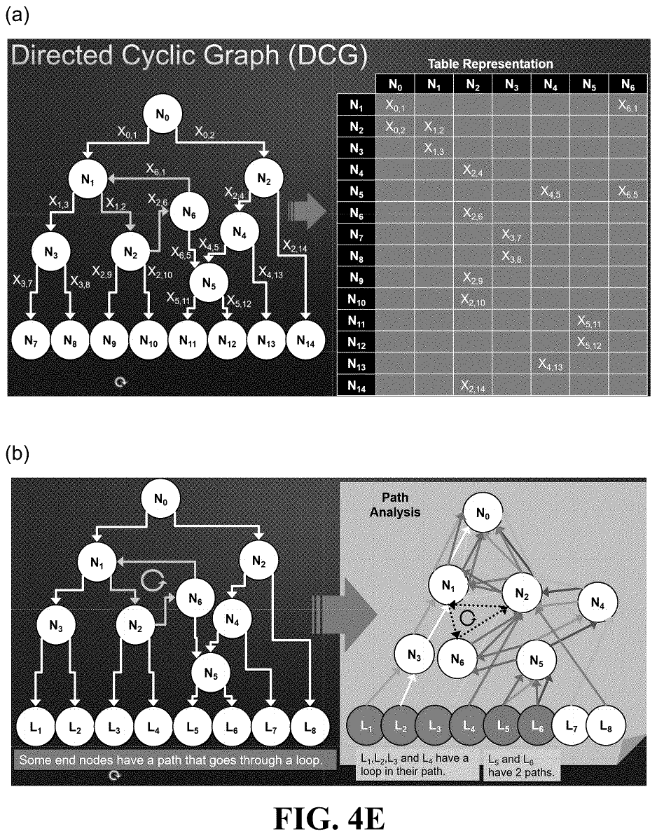

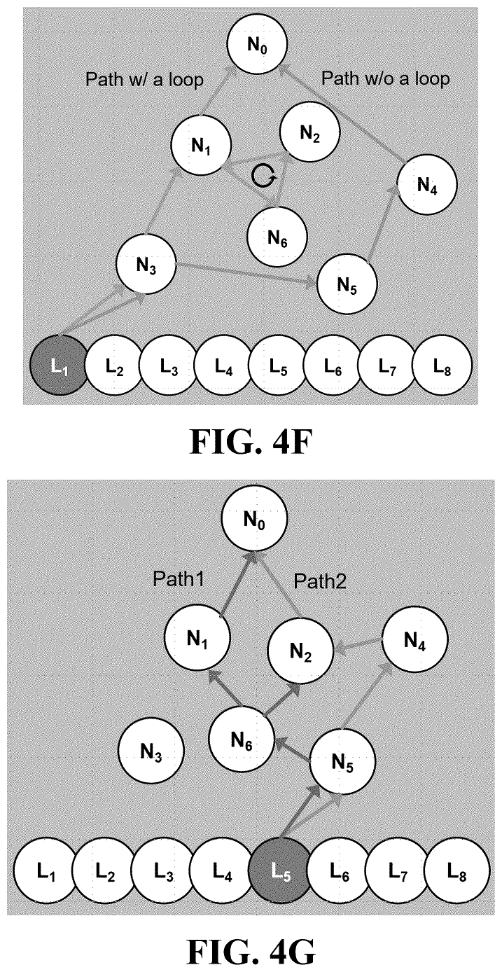

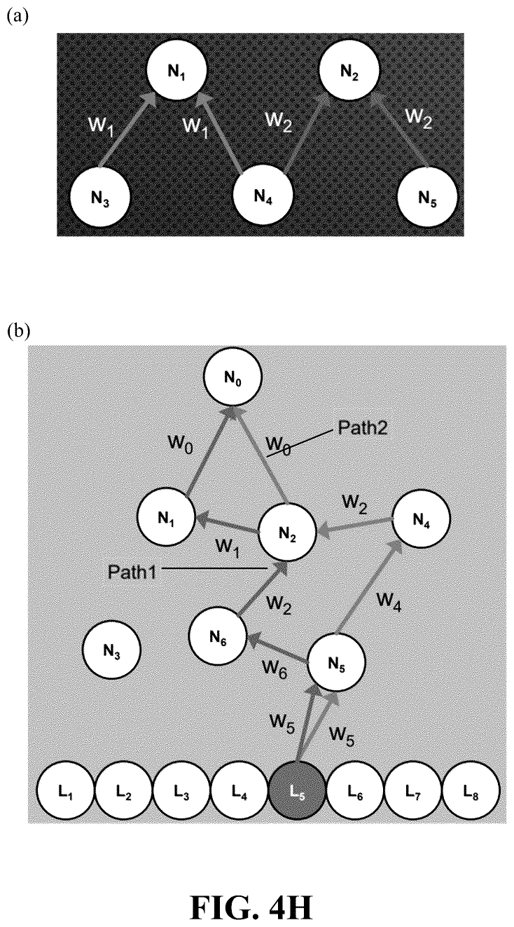

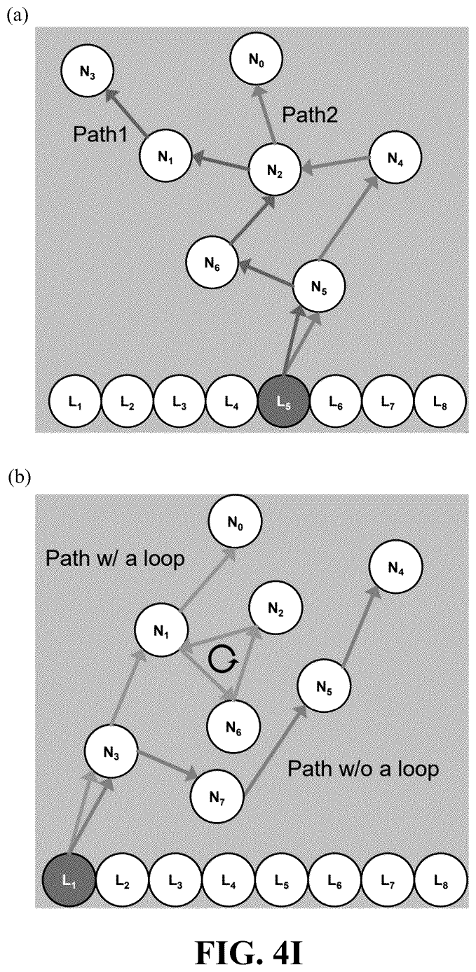

FIG. 4A shows an example diagram showing various embodiments of a data computation method, including graphs, directed cyclic graphs (DCGs), directed acyclic graphs (DAGs), weighted DCGs, weighted DAGs, and decision trees.

FIG. 4B shows an example directed graph configuration of a computation method. In FIG. 4A, Nᵢ, Nⱼ, and Nₖ are nodes of the directed graph. Node Nᵢ is a root node because it has no incoming edges. Nodes Nⱼ and Nₖ are leaf (or end) nodes because each has no outgoing edge. X_{i,j} is an edge from node i to node j, and X_{i,k} is an edge from node i to node k. Each edge X_{i,j} and X_{i,k} can be a rule, a formula, a weight, a value, a flag, or a tag.

When multiple types of edges are used in a graph configuration, the graph becomes multi-graph. If graphs have weights assigned to their edges, they are weighted graphs.

After generating a data computation method as a graph, to reach a decision outcome (i.e., end node), the exemplary system can employ a path-finding (i.e., path analysis) operation to find a path from a decision outcome (i.e., end node, leaf node) to a root node. Unlike decision process in a data computation method, path analysis is a reverse flow from end (decision outcome) to beginning (root node).

FIG. 4C shows an example decision tree configuration for the data computation method. Subpanel (a) shows the decision tree configuration and its associated table representation. Subpanel (b) shows all paths from the end nodes L₁-L₈ of the decision tree to the root node NO.

FIG. 4D shows an example directed acyclic graph (DAG) configuration for the data computation method. Subpanel (a) shows the DAG configuration and its associated table representation. Subpanel (b) shows all paths from the end nodes L₁-L₈ of the DAG to the root node NO and.

FIG. 4E shows an example directed cyclic graph (DCG) configuration for the data computation method. Subpanel (a) shows the DCG configuration for the data computation method and its associated table representation. Subpanel (b) shows all paths from the end nodes L₁-L₈ of the DCG to the root node NO.

In subpanel (b), when a path enters a loop (i.e., cycle), the exemplary system can detect that a node in the path is revisited (i.e., visited twice) and (i) display a warning message on the user interface, (ii) pause the operation and require human intervention, or (iii) update the values of nodes in the loop. The exemplary system can detect and evaluate loops (i) using a loop-detection algorithm (e.g., depth-first search, breadth-first search) or (ii) using a trained artificial intelligence (AI) model.

Path analysis. A path-finding (i.e., path analysis) operation can comprise (i) identifying data dependencies within a transaction, (ii) identifying end nodes with multi-paths in a DAG, (iii) identifying end nodes with loops in a DCG, (iv) identifying alternative paths for an end nodes in a DCG, (v) identifying the most efficient path (e.g., shortest path, least-weighted path) for a decision outcome in a graph, (vi) identifying dominant root nodes (e.g., a root node that gets to a decision outcome (i.e., end node)) most efficiently, and/or (vii) comparing and evaluating alternative graphs. Path analysis may be performed by the offline tool during the setup of a new transaction and data computation method. Path analysis may also be performed by the data computation engine (e.g., 128) during runtime when an update is provided by the user that changes a value of the data element in a data computation method 106. To this end, the path analysis may be performed by the data computation engine (e.g., 128) to determine if the workflow or decision logic has to be changed, i.e., invoke new tasks or update tasks.

Multi-path analysis (MPA). The exemplary system can identify multiple paths to reach a decision outcome (i.e., end node) from the root node. To identify the best paths to reach the outcome, the exemplary system can further employ a multi-path evaluation operation. FIG. 4F shows an example DCG having multiple paths between an end node L₁ and the root node No, wherein there is a path without a loop.

FIG. 4G shows an example multi-path analysis method #1. In FIG. 4G, two paths (e.g., path 1 and path 2) connect the root node N₀ and the end node L₅. The exemplary system, using the MPA method #1, chooses path 2 because path 2 requires the least number of steps, i.e., includes the least number of edges. The MPA method #1 does not work if path 1 and path 2 have the same number of steps.

FIG. 4H shows an example multi-path analysis method #2. In subpanel (a), the MPA method #2 assigns weight w₁ to every edge leading to node N₁ and weight w₂ to every edge leading to node N₂. A weight (e.g., w₁, w₂) can be a reward value (e.g., trustworthiness, verified/non-verified status, predictive power, etc.) or a penalty value (e.g., information collection cost, verification difficulty, fraud tendency, etc.). Table 3 shows example definitions of weight types (e.g. reward value, penalty value, net value) used in the exemplary system.

TABLE 3

| Weight type | Example Definition |
|---|---|
| Reward value | If Asset can be verified more easily than Income does, then w(Asset) > |
| | w(Income) (i.e., weight of Asset is larger than weight of Income). |
| | If Credit Score is more predictable than Age, then w(Credit Score) > |
| | w(Age). |
| Penalty value | If Asset information is more costly to collect/verify than Credit Score, |
| | then w(Asset) > w(Credit Score). |
| | If Income is self-reported and Credit Score is from a third party, then |
| | w(Income) > w(Credit Score). |
| Net value | Net value can be defined as: w^{Net}(Nodeᵢ) = w^{Benefit}(Nodeᵢ) − |
| | w^{Penalty}(Nodeᵢ). |

In subpanel (b), the exemplary system uses MPA method #2 to find the best path between the root node N₀ and the end node L₅. As shown, path 1 and path 2 weights can be calculated per Equations 2 and 3, respectively.

$${w}_{p a t h 1} = {w}_{0} + {w}_{1} + {w}_{2} + {w}_{6} + {w}_{5} \qquad ( Eq . 2 )$$

$${w}_{p a t h 2} = {w}_{0} + {w}_{2} + {w}_{4} + {w}_{5} \qquad ( Eq . 3 )$$

The exemplary system can choose a path with the highest total reward, the lowest total penalty, or the highest total net value using the path 1 and path 2 weights calculated per Equations 2 and 3.

The exemplary system can also implement an MPA method using dynamic weights (e.g., weight changes when the verified status of a node is updated) generated by a trained AI model. The exemplary system can use the trained AI model to analyze and predict the best next moves (e.g., backward reduction, strategies in the Game of Go) on a path between a root node and an end node (i.e., decision outcome).

The above-discussed path-finding and multi-path analysis operations, employed by the exemplary system, can also be used in multi-root-node graphs. FIG. 4I shows example multi-root-node graphs having multiple paths between multiple root nodes and leaf nodes. Specifically, in subpanel (a), the acyclic multi-root-node graph comprises two paths (e.g., paths 1-2), wherein path 1 connects the root node N₃ with the leaf node L₅ and path 2 connects the root node N₀ with the leaf node L₅. In subpanel (b), the cyclic multi-root-node graph comprises two paths, wherein the path with a loop connects the root node N₀ with the leaf node L₁, and the path without a loop connects the root node N₄ with the leaf node L₁.

Additional graph definitions. Table 4 shows an example graph evaluation operation.

TABLE 4
For Graph(i): w_{graph}(i) = w_{path,EndNode1} (i,1) + w_{path,EndNode2}(i,2) +...+w_{path,EndNodeK}(i,K)
Use w_{graph}(i) to evaluate multiple graphs, where i = 1, 2, ... , I
Evaluation criteria: most predictive power, most efficient, least penalty, and/or least complex.

End nodes (i.e., leaf nodes, decision outcomes) can be defined by one or multiple data elements. A possible decision outcome can only be represented by one end node.

Table 5 shows example data elements and their strict end nodes in a graph used by the exemplary system.

TABLE 5

| | |
|---|---|
| Data elements (D) | D₁ = Decision = [Approved, Rejected] |
| | D₂ = Loan Terms = [6 months, 1 year, 5 years, 10 years, 15 years] |
| | D₃ = Loan Amount = [50k, 100k, 500k, 1000k] |
| Single data | For single data element D₁, there are 2 possible end nodes (N): |
| element | N₁ = [Approved] |
| | N₂ = [Rejected] |
| Multiple data | For multiple data elements D₁, D₂, and D₃, there are 40 (2 × 5 × 4) possible |
| elements | end nodes (N): |
| | N₁ = [Approved, 6 months, 50k] |
| | N₂ = [Approved, 1 year, 100k] |
| | . . . |
| | Ni = [Approved, 5 years, 500k] |
| | . . . |

Table 6 shows example data elements and their flexible end nodes in a graph used by the exemplary system.

TABLE 6

| | |
|---|---|
| Data elements (D) | D₁ = Decision = [Approved, Rejected] |
| | D₂ = Loan Terms = [6 months, 1 year, 5 years, 10 years, 15 years] |
| | D₃ = Loan Amount = [50k, 100k, 500k, 1000k] |
| Single data | For single data element D₁, there are K possible end nodes, wherein K is |
| element | not finite: |
| | N₁ = [Approved] |
| | N₂ = [Rejected] |
| | N₃ = [Approved] |
| | N₄ = [Rejected] |
| | . . . |
| | Nₖ = [Rejected] |
| Multiple data | For multiple data elements D₁, D₂, and D₃, there are M possible end |
| elements | nodes, wherein M is not finite: |
| | N₁ = [Approved, 6 months, 50k] |
| | N₂ = [Approved, 1 year, 100k] |
| | N₂ = [Rejected, —, —] |
| | . . . |
| | Ni = [Approved, 1 year, 100k] |
| | . . . |
| | N_{M} = [Approved, 6 months, 50k] |

### Example Decision-Making and Task Management System

A decision-making and task management system 112 is now described, e.g., as that implemented in FIG. 1B.

Task. A basic unit of work by users can be divided and arranged into definable groups of definable/configurable tasks. Each transaction type utilizes a set of data elements and, therefore, is assigned a set of tasks related to the transaction type's intended usage and the necessity of having the tasks to make changes to the data elements.

Even though human users can perform tasks, tasks can also be automated by machines or interfaced with other external systems to fully or partially perform steps on other systems to complete the tasks.

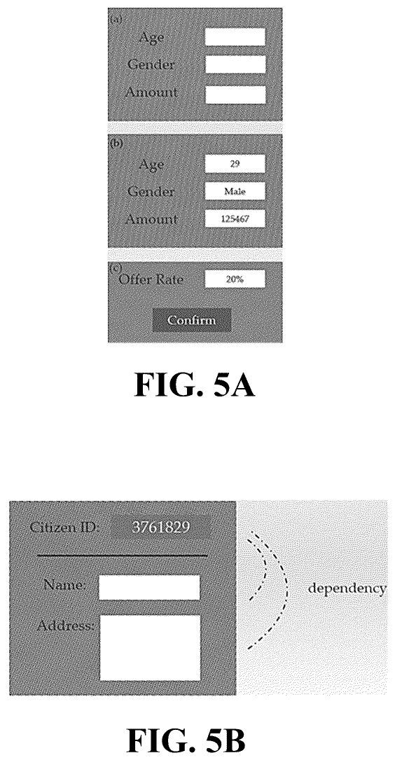

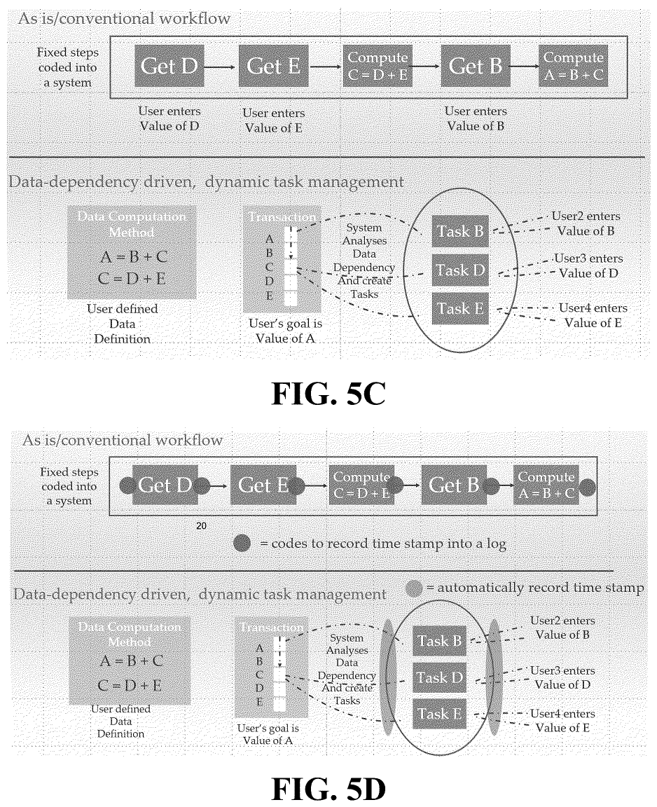

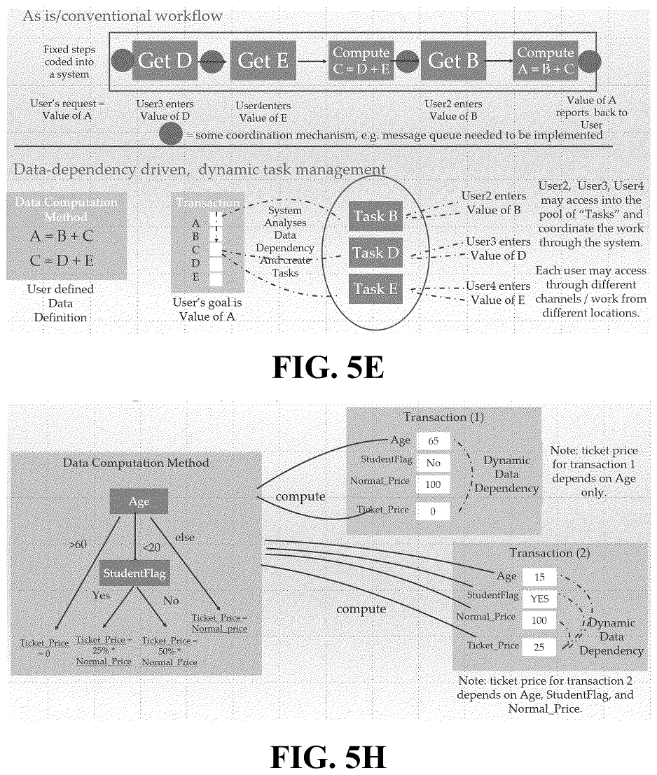

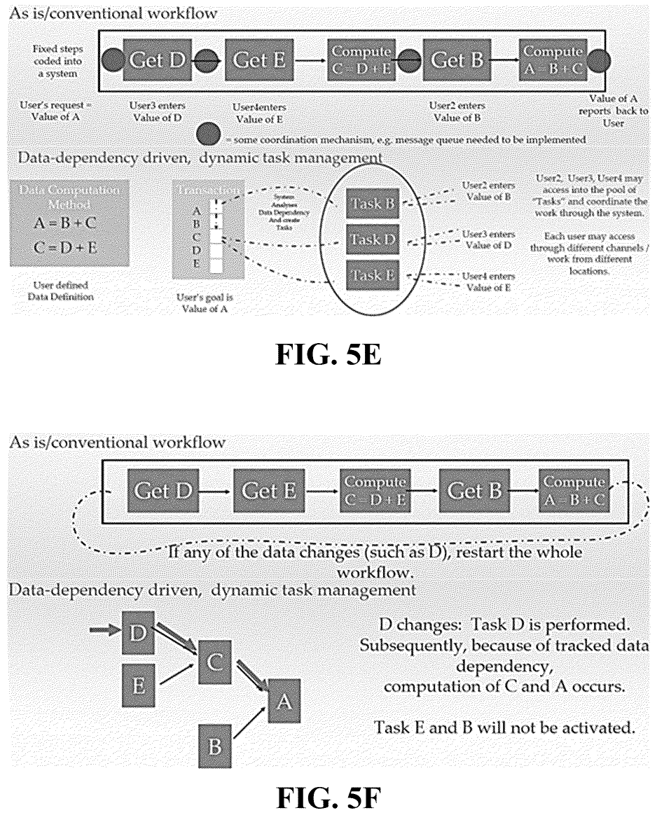

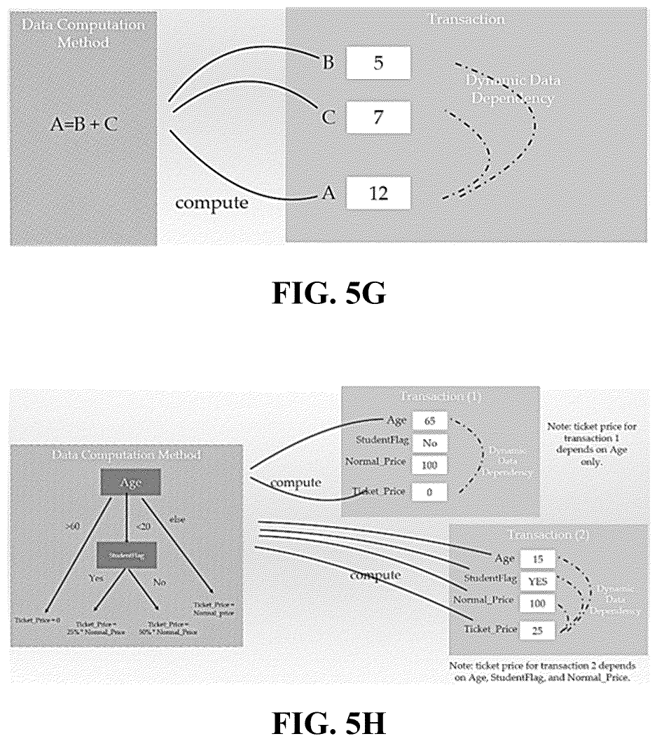

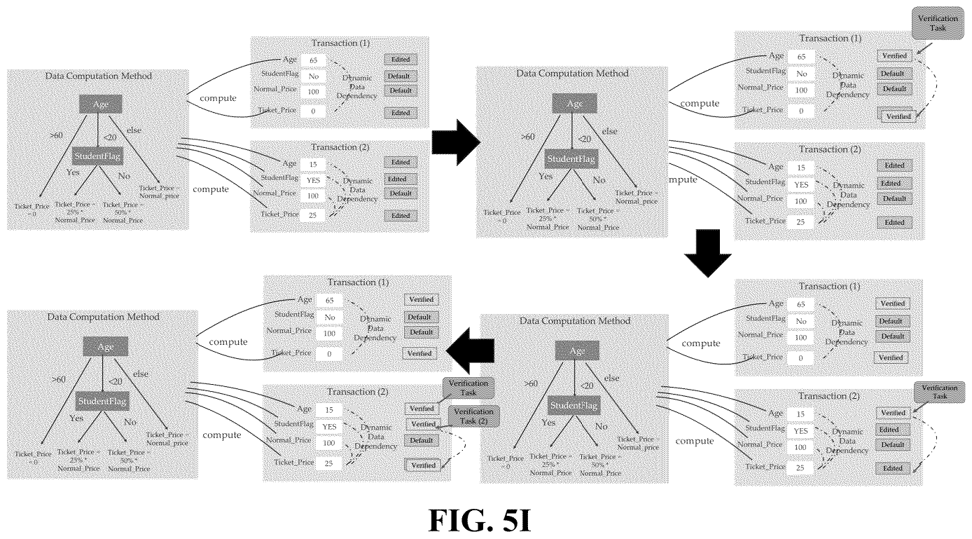

FIG. 5A shows an example task defined as an input screen (e.g., for data entry), an editing prompt, or an action prompt. In subpanel (a), task is an input screen (data entry) for capturing a set of data elements (e.g., age, gender, amount). In subpanel (b), task is for a single data element or a subset of all data elements used in the transaction type. In subpanel (c), task is an action of validating or authorizing (e.g., confirm), thereby changing the status of data elements.

Task can also define required data (i.e., prerequisite) to perform the task and create data dependency relationships between data elements in the transactions that utilize the task. FIG. 5B shows an example task requiring data to create data dependency relationships between data elements.

Task management. With the tasks and data computation methods defined, the task management system can analyze data dependencies, keep track of dynamic changes of data dependencies, and manage the tasks to fulfill the user's goals (i.e., requests). With the dynamism of changes of data and data dependencies, the exemplary system can coordinate, distribute, and present the required tasks to multiple groups of users across many user interface channels while tracking all these activities to contribute to the final computation of user's goals.

Unlike the conventional design of a computer system where fixed workflows or flowcharts need to be coded into the system for a specific purpose, the exemplary system can (i) utilize the discovery of dynamic data dependencies through the definition of atomic tasks and data computation methods and (ii) derive the necessary processes comprising of a series of tasks to achieve the end goal. Tasks can be executed in parallel when there are no data dependencies that prevent parallel execution.

For a transaction, a group of tasks may require to be handled to achieve a user's goal, represented by the realization of a value of some data elements. This group of tasks can be handled by various human agents/users or connected interfaced systems to perform the tasks. This group of tasks can be visualized as a pool of tasks (shown as task pool in FIG. 1B) where a group of agents may look into the pool and pull tasks from the pool to complete, i.e. as a “scrum” team storming force together to get the work done.

With definitions of how data can be computed, the exemplary system can derive the processes without specifying a fixed workflow. FIG. 5C shows an example conventional workflow and workflow configured by the exemplary system (shown as data-dependency driven, dynamic task management).

Likewise, tasks and other more complex data computation methods create data dependencies among data elements. Subsequently, the exemplary system tracks the data dependencies and manages the required processes, i.e., the task pool to be completed. Because of the dynamic nature of data dependencies, which can change whenever there are changes in the values of some data elements, the exemplary system manages the task pool accordingly.

Access rights. Tasks can be controlled by an authorization matrix, which defines the relationship between user-role, role authorization, and applicable conditions defined on the values of some data elements. The authorization matrix can manage which groups of users have access to and can perform what types of tasks.

Time stamp. Time stamps can be recorded for every step involving a task from creation, being fetched by a user, to completion. This occurs automatically by the task management operation in the exemplary system. Code does not need to be inserted to record time stamps of a task during the exemplary system as in conventional workflow. FIG. 5D shows example time stamps recorded in a conventional workflow and a workflow configured by the exemplary system.

Multi-users and multi-channels. Both synchronous and asynchronous orchestration of tasks among many users across different channels can be simple and native in the task management of the exemplary system. FIG. 5E shows an example multi-user and multi-channel configurations in a conventional workflow and a workflow configured by the exemplary system.

Resilience and ease of recovery. Because the task pool can be derived from data dependencies and the value of data elements in the transactions, the resilience of the exemplary system and the process of recovery from system failure can depend only on the data in the transactions. Since there is no queuing mechanism or immediate steps in the middle of a workflow to safeguard against, as in a conventional implementation (of workflows), recovery can be simpler.

In the case of direct intervention, when there is a fault in a system, the intervention can be a direct change to some values of data elements. There is no intermediate step or queuing to reverse or clear to change the path of execution of a transaction.

Incremental changes. Since data dependencies can be tracked, incremental changes through chains of computation can be achieved without restarting the whole workflow in the exemplary system. FIG. 5F shows an example change to a conventional workflow and workflow configured by the exemplary system.

Data dependency. Both data computation methods and tasks, when applied, can create dynamic data dependencies between data elements in a transaction. FIG. 5G shows an example dynamic dependency between a data computation method and a transaction.

Data dependency can be dynamic for each transaction. FIG. 5H shows an example data dependency configured to be dynamic for each transaction.

Similar to data computation methods, tasks that are executed result in changes in the value of data elements and data dependencies. Data dependency may differ from one transaction to another, even though transactions are of the same type. Data dependency of a single transaction can also change during the lifetime of a transaction. Changes in the value of data elements, by tasks or data computation methods, may result in ripples or cascading effects on changes of value of data elements and data dependencies in the transaction.

Table defines data dependency. Besides other discrete structures, a computation or a relationship between data elements can be defined by a table of possible values of many data elements. The exemplary system can look for one-to-one or many-to-one to derive data dependencies and implicit data computation methods. For a one-to-one relationship, data dependency can go both ways between data elements on the table. For many-to-one, the data element on the “one” side of the relationship can depend on the data elements on the “many” side.

For example, suppose data element “A” is a unique city name and data element “B” is the province name. The table that defines the relationship can be a list of cities and the corresponding province where the city resides. By listing the possible values of A and B, the dependency is also defined, which means that whenever the value of A (city) is given or changed by the user, the value of B (province) can be computed by looking up from the table. With the exemplary system, all these can be derived without programming, coding, or explicit workflow implementation. Vice versa, whenever the value of B (province) is given or changed by the user, all the possible values of A (city) can be computed as a list for the user to pick or select within the list.

Dependency handling. Tracking dependencies can be done in many ways. One example is propagating changes through a directed graph. One common software pattern can be an “Event Driven System using Event Sourcing” pattern, “Change Data Capture (CDC)” pattern, or Pub/Sub (Publisher/Subscriber) pattern. Common implementations can include (i) database level, (ii) stream processing, and (iii) message brokers.

Status of Data Elements in Transactions. Status of Data Elements can keep track of data changes that occur by users entering or editing the data or by automatic computation of the data through dependencies. The status of data elements can be one of these possible status values: new/default, edited, or verified. The status of data elements can be cascaded through data dependencies and data computation methods. A data element derived from at least one of the other edited data elements can also end up with the edited status.

The status of a data element can turn from edited to verified in one of the two conditions: (i) completion of a task defined to be a verification task of that data element, or (ii) the status of all the data elements that are prerequisite to the computation of the data element is either verified or new/default, and the data element is not null or defined to be nullable. Through data dependencies, all prerequisite data elements should be verified before the status of the dependent data element can be verified.

FIG. 5I shows the cascading status of data elements via data dependency in the exemplary system.

Status of Data Elements can allow computation of value to be carried out without waiting for the validation of the value of data elements, which can be useful in many applications such as the preliminary/pre-approval process, e.g., in loan origination or in most request-approval processes. When applied to the medical diagnosis process, the status of data elements can facilitate using “assumptions” for the disease diagnosis to further explore the possibilities and gather more data while waiting for lab results to verify the assumed data.

Using verification task to model authorization process. With the exemplary system, users can define some tasks to be verification tasks and combine them with defining access rights to model any authorization process. In FIG. 5I, the value of the data element “NormalPrice” is set at default to be 100, and thus, the status of “NormalPrice” is default. Without anyone touching the value of “NormalPrice”, if other data elements are verified, the “TicketPrice” can be computed and finally verified.

However, if a user requests a special treatment of “NormalPrice” and needs to change the value to 50, the status of “NormalPrice” becomes edited and therefore requires a verification task of the data element “NormalPrice”. The access right of such verification task of “NormalPrice” is set to only be accessible to a privileged supervisor user, which models the authorization process of the supervisor. The final status of “TicketPrice” can turn into verified” only when the supervisor completes the verification task of the value 50, which is made onto the data element “NormalPrice”.

Data computation method (also referred to as discrete structure) generation. A data computation method (e.g., decision tree, directed graph, table, etc.) can be generated to represent the organization's desired decision-making process. Table 6 shows the exemplary embodiments of data computation method generation.

TABLE 6

| Data Computation | |
|---|---|
| Method generation | |
| approach | Description |
| Heuristic Approach | The heuristic approach collects institutional knowledge from senior |
| | officers or a group of individuals involved in the decision-making |
| | process. Organizational operating procedures or policy documents |
| | may also be consulted. A data computation method is iteratively |
| | created to map out the desired decision-making process. |
| Intelligent Approach | Intelligent approach, whereby a machine learning processing tool, |
| | such as CART, is applied to create an optimized data computation |
| | method using training data. |
| Hybrid Approach | Hybrid approach: The data computation method generated with the |
| | intelligent approach can be an initial map that is further augmented |
| | by iterative refinements to meet certain criteria, e.g., regulatory |
| | compliance, organization requirements, etc. |

The exemplary system's intelligent data computation method generation (i.e., graph generation) approach is data-driven, using a training set of transactions. This approach can be applied to meet certain design objectives, such as optimizing the predictive power of decision outcomes, e.g., disapproving transactions with high default risk. Both input and computed data elements can be used in the training steps of the intelligent approach. In the training data, all computed data elements can be populated with the values of input data elements. Multiple machine-learning processing tools exist for such purposes, including a decision tree generated with Classification And Regression Tree (CART).

Intelligent approach can provide several benefits, including but not limited to (i) optimizing the predictive power of decision outcomes, (ii) optimizing the discriminatory power of the discrete structure based on information in data elements, (iii) defining, based on training data, the cut-offs for branching (or splitting) of numerical data elements, (iv) defining, based on training data, the placements of data elements within the discrete structure, (v) uncovering new branches resulting in a discrete structure that is adaptive to new data, i.e. continual learning, and (vi) pruning unnecessary branches within the discrete structure resulting in more efficient decision-making processes.

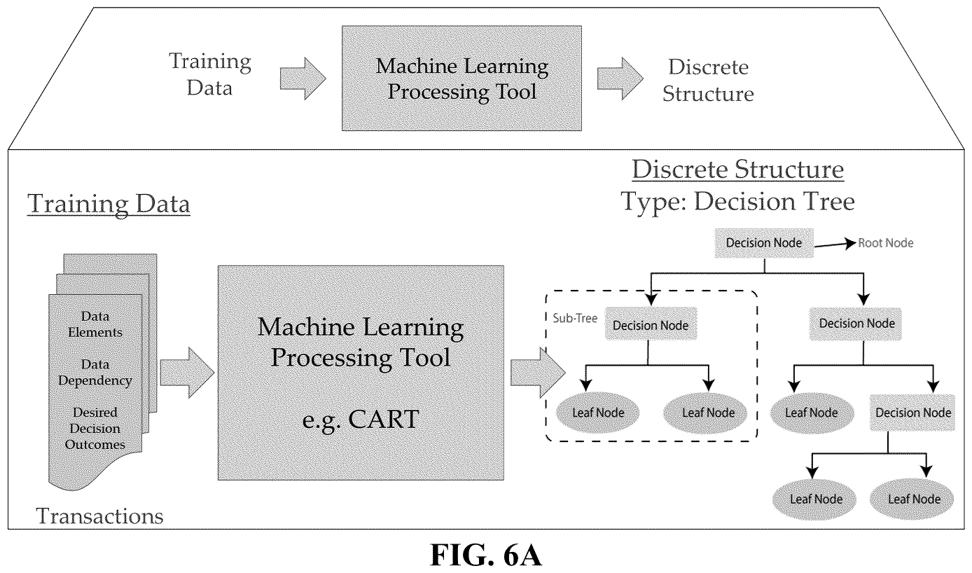

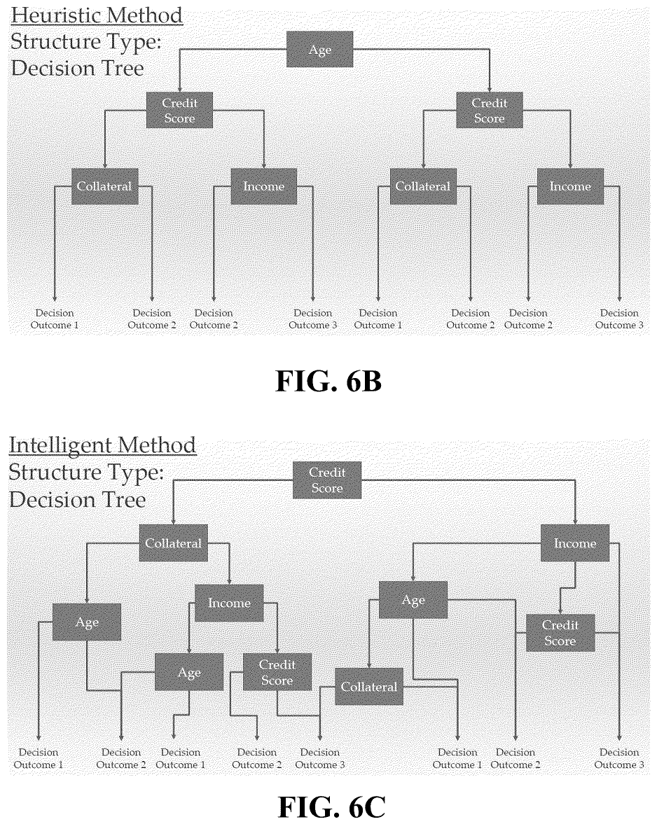

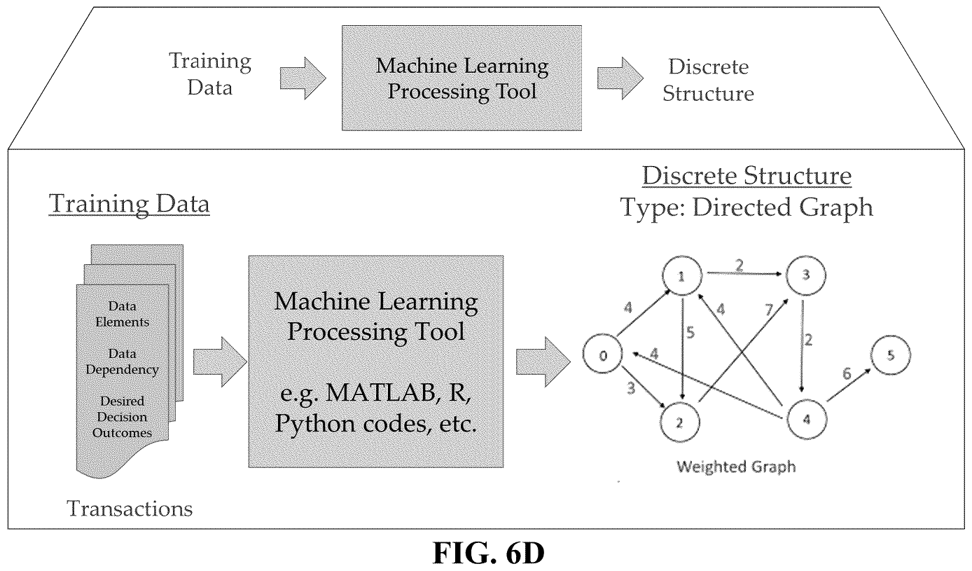

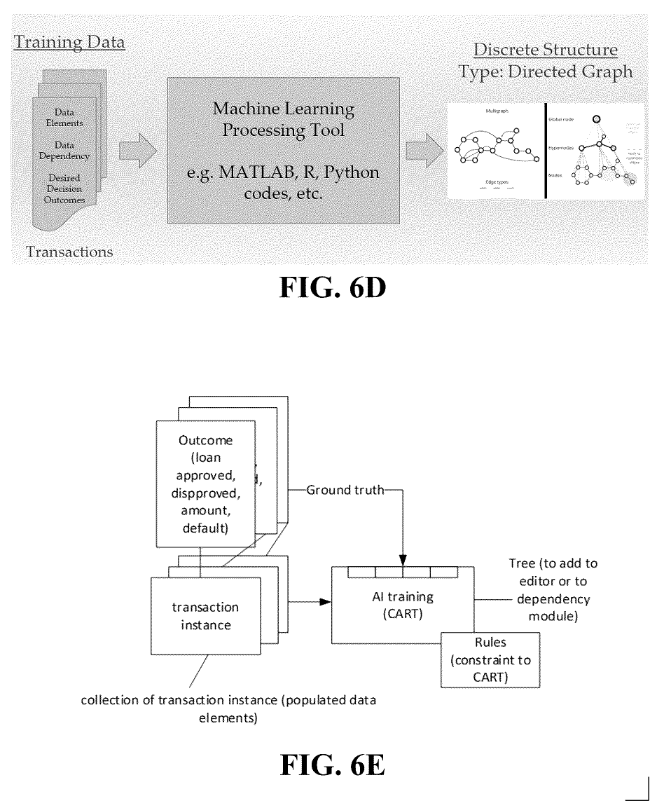

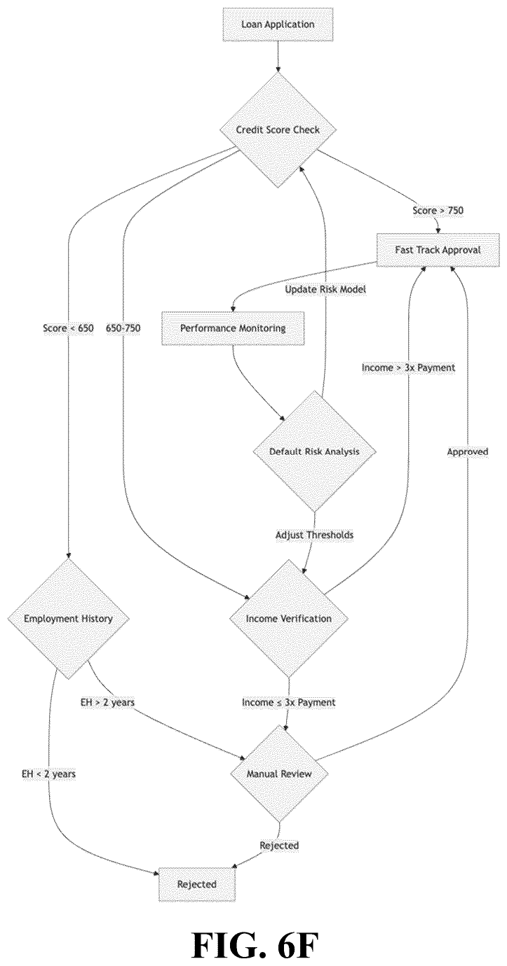

Machine learning (ML) is a data-driven analytical engine. There are many ML processing tools (i.e., ML modules), but the exemplary system only uses the ones configured to create data computation methods (e.g., directed graphs, decision trees, tables, etc.) Of the selected ML processing tools, (i) they are not black-box because generated data computation methods (as outputs of the ML process) are intuitive (i.e., reviewed and understood), (ii) decision flows through the generated data computation methods are transparent and can be validated, and (iii) there are no hidden decision nodes. FIG. 6A shows an example ML processing tool used in the exemplary system to generate a decision tree.

FIG. 6B shows an example decision tree generated by the conventional generation (i.e., heuristic approach) method.

FIG. 6C shows an example decision tree generated by the intelligent generation method (i.e. intelligent approach) employed by the exemplary system.

FIG. 6D shows an example ML processing tool used in the exemplary system to generate a directed graph.

FIG. 6E shows an example AI training system for the ML processing tool using CART. The training system can employ past outcomes (e.g., loan approval/disapproval, approved amount, default, etc.) as labels/ground truths for training using user-provided input and documents or a transaction instance encoding the same as the input.

FIG. 6F shows an example directed graph.

### Example Set of Rules

The set of rules employed by the exemplary system can be configured to express business logic in a structured, human-readable format that bridges the gap between business users and technical implementation, facilitating organizations to centralize decision logic, manage, audit, and modify business rules more easily.

Table 7 shows the core syntaxes of the exemplary set of rules.

TABLE 7

| Core syntax | Description |
|---|---|
| Rule | Rule Structure |
| structure | RULE “[rule_name]” |
| | WHEN |
| | [conditions] |
| | THEN |
| | [actions] |
| | PRIORITY [integer] |
| | DESCRIPTION “[description]” |
| | END |
| Rule set | RULESET “[ruleset_name]” |
| | DESCRIPTION “[description]” |
| | RULES |
| | [rule_1] |
| | [rule_2] |
| | ... |
| | EXECUTION_STRATEGY [FIRST_MATCH \| |
| | ALL_MATCHING] END |
| Condition | Comparison operators: ==, !=, >, >=, <, <= |
| | Logical operators: AND, OR, NOT |
| | Functions: contains( ), startsWith( ), endsWith( ), |
| | isEmpty( ), matches( ) |
| | Value access: object.attribute |
| | Collection access: list[index] |
| Action | Assignment: SET object.attribute = value |
| | Function calls: CALL functionName(parameters) |
| | Rule flow: EXECUTE_RULESET “ruleset_name” |
| Variable and | Primitive types: String, Number, Boolean, Date |
| Data type | Complex types: Object, List, Map |
| | Type declaration: var_name: Type |
| Temporal logic | WITHIN [time_period] OF [event] |
| | AFTER [event] FOR [time_period] |
| | BEFORE [event] |
| Contextual | currentDate( ) |
| functions | timeElapsedSince(event) |
| | average(collection.attribute) |
| | sum(collection.attribute) |
| Decision | DECISION_TABLE “[table_name]” |
| tables | INPUT |
| | [input_1]: [type_1] |
| | [input_2]: [type_2] |
| | ... |
| | OUTPUT |
| | [output_1]: [type_1] |
| | [output_2]: [type_2] |
| | ... |
| | RULES |
| | \| [input_1] \| [input_2] \| ... \| [output_1] \| |
| | [output_2] \| ... \| |
| | \|-----------\|-----------\|-----\|------------\|------------\|-----\| |
| | \| value \| value \| ... \| value \| value \| ... \| |
| | \| value \| value \| ... \| value \| value \| ... \| |
| | DESCRIPTION “[description]” |
| | END |
| External data | DATA_SOURCE “[source_name]” |
| sources | TYPE [SQL \| REST \| SERVICE] |
| | CONNECTION “[connection_string]” |
| | CACHE_DURATION [time_period] |
| | RETRY_POLICY [policy] |
| | END |
| Versioning | RULESET “[ruleset_name]” VERSION “[version]” |
| | EFFECTIVE_FROM [date] |
| | EFFECTIVE_UNTIL [date] |
| | ... |
| | END |
| Audit and | RULE “[rule_name]” |
| tracing | ... |
| | AUDIT LEVEL [NONE \| BASIC \| DETAILED] |
| | TRACE [TRUE \| FALSE] |
| | END |
| Comments | // Single line comment |
| | /* |
| | Multi-line |
| | comment |
| | */ |
| Tags and | RULE “[rule_name]” |
| Categories | ... |
| | TAGS [“tag1”, “tag2”, ...] |
| | CATEGORY “[category]” |
| | END |

### Experimental Results and Additional Examples

Development was performed develop and evaluate the exemplary system and method for an automated or semi-automated decision-making platform.

Table 8 shows the applications for the exemplary system.

TABLE 8

| | | | | | Decision |
|---|---|---|---|---|---|
| Organization | Use case | Transaction | Data Element | Task | outcome |
| Lending | Loan | Loan | Credit Score, | Authenticate | Approve, |
| Institutions | Origination | Applications | Age, Income, | identity, verify | Decline, Loan |
| | | | Collateral, | supporting | Amount, Loan |
| | | | Loan Amount, | documents, | Term, Interest |
| | | | Loan Term | assess | Rates |
| | | | | delinquency | |
| | | | | risk | |
| Insurance | Insurance | Life Insurance | Age, Smoke | Authenticate | Approve, |
| Companies | Underwriting | Applications | History, | identity, verify | Decline, |
| | | | Health | health history, | Insurance |
| | | | Report, | perform | Premium |
| | | | Lifestyle, | actuarial risk | |
| | | | | analysis | |
| County | Permit | Building Permit | Street | Verify address, | Approve, |
| Building | Approval | Applications | Address, | review | Reject, |
| Departments | | | Floor Plan, | blueprint, | Request for |
| | | | Blueprint/ | review town | more |
| | | | Drawings, | codes; review | information |
| | | | Construction | safety | |
| | | | Materials | regulations | |
| Hospitals | Patient | Patient Visits | Patient ID, | Authenticate | Routing to an |
| | Check-in | | Visit Purpose, | patient | appropriate |
| | | | Symptoms, | identity, take | treatment unit |
| | | | Vital Sign | vital sign | |
| | | | Measures, | measurements, | |
| | | | Current | conduct | |
| | | | Medications, | preliminary | |
| | | | Allergies | patient | |
| | | | | interview | |
| Lending | Collateral | Collateral Files | Loan ID, | Verify loan ID, | Validated, |
| Institutions | Management | | Loan | review | Loan under |
| | | | Principal | collateral | collateralized |
| | | | Balance, | condition, | |
| | | | Collateral | assess LTV | |
| | | | Type, | (Loan-To- | |
| | | | Collateral | Value) | |
| | | | Condition, | | |
| | | | Collateral | | |
| | | | Market Value | | |
| Insurance | Claim | Auto Accident | Vehicle ID, | Verify vehicle | Approve, |
| Companies | Management | Claim Filings | Accident | ID, review | Request for |
| | | | Date, Police | police report, | more |
| | | | Report, | assess damages | information |
| | | | Damages | | |
| Research | Grant | Grant Proposals | Submitted | Verify data, | Approve, |
| Funding | Proposal | | Institution, | review | Reject |
| Institutions | Review | | Research | proposed | |
| | | | Type, Grant | research | |
| | | | Request, | method, assess | |
| | | | Research | research | |
| | | | Method, | timeline | |
| | | | Research | | |
| | | | Schedule | | |
| Pharmaceutical | Drug | Drug | Program ID, | Verify data, | Abort, Ready |
| Companies | Development | Development | Drug Name, | initiate a | for FDA |
| | Management | Programs | Potential | research step, | |
| | | | Treatment, | assess test | |
| | | | Research | results | |
| | | | Steps | | |

Table 9 shows the example applications for the exemplary set rules (e.g., decision rules language (DRL)).

TABLE 9

| Example application | Description | |
|---|---|---|
| Load application | RULESET “LoanApplicationProcessing” VERSION “1.2” | |
| processing rules | DESCRIPTION “Rules for evaluating loan applications” | |
| | EXECUTION_STRATEGY ALL_MATCHING | |
| | RULE “CreditScoreEvaluation” | |
| | WHEN | |
| | application.creditScore >= 750 | |
| | THEN | |
| | SET application.riskCategory = “Low” | |
| | SET application.interestRate = 3.75 | |
| | PRIORITY 100 | |
| | DESCRIPTION “Assign low risk category for excellent credit | |
| | scores” | |
| | END | |
| | RULE “MediumCreditRisk” | |
| | WHEN | |
| | application.creditScore >= 650 AND | |
| | application.creditScore < 750 | |
| | THEN | |
| | SET application.riskCategory = “Medium” | |
| | SET application.interestRate = 5.25 | |
| | PRIORITY 90 | |
| | DESCRIPTION “Assign medium risk category for good credit | |
| | scores” | |
| | END | |
| | RULE “HighCreditRisk” | |
| | WHEN | |
| | application.creditScore < 650 | |
| | THEN | |
| | SET application.riskCategory = “High” | |
| | SET application.interestRate = 7.50 | |
| | PRIORITY 80 | |
| | DESCRIPTION “Assign high risk category for lower credit scores” | |
| | END | |
| | RULE “DebtToIncomeRatio” | |
| | WHEN | |
| | application.debtToIncomeRatio > 0.43 | |
| | THEN | |
| | SET application.eligibility = “Rejected” | |
| | SET application.rejectionReason = “Debt to income ratio too high” | |
| | PRIORITY 120 | |
| | DESCRIPTION “Reject applications with DTI above lending | |
| | threshold” | |
| | END | |
| | RULE “LoanAmountLimit” | |
| | WHEN | |
| | application.requestedAmount > 500000 | |
| | THEN | |
| | EXECUTE_RULESET “EnhancedVerification” | |
| | PRIORITY 110 | |
| | DESCRIPTION “Trigger additional verification for large loan | |
| | amounts” | |
| | END | |
| | END | |
| E-commerce pricing | RULESET “PricingAndDiscounts” | |
| and discount rules | DESCRIPTION “Dynamic pricing and discount application rules” | |
| | RULE “SeasonalDiscount” | |
| | WHEN | |
| | currentDate( ) WITHIN PERIOD(“11/15/2024”, “12/31/2024”) | |
| | AND NOT product.category == “Electronics” | |
| | THEN | |
| | SET product.discountPercentage = 15 | |
| | CALL applyDiscount(product) | |
| | PRIORITY 50 | |
| | TAGS [“seasonal”, “holiday”, “discount”] | |
| | DESCRIPTION “Apply holiday season discount to non-electronic | |
| | items” | |
| | END | |
| | RULE “VolumeDiscount” | |
| | WHEN | |
| | cart.itemCount >= 5 AND | |
| | cart.items.allMatch(i -> i.category == “Books”) | |
| | THEN | |
| | SET cart.additionalDiscount = 10 | |
| | SET cart.discountType = “Volume” | |
| | PRIORITY 40 | |
| | DESCRIPTION “Apply additional discount for bulk book | |
| | purchases” | |
| | END | |
| | RULE “LoyalCustomerDiscount” | |
| | WHEN | |
| | customer.membershipLevel == “Gold” AND | |
| | customer.accountAgeInYears >= 2 | |
| | THEN | |
| | SET customer.eligibleForSpecialOffers = true | |
| | SET order.discountPercentage = MAX(order.discountPercentage, | |
| | 12) | |
| | PRIORITY 60 | |
| | AUDIT LEVEL DETAILED | |
| | DESCRIPTION “Apply loyalty discount for long-term gold | |
| | members” | |
| | END | |
| | DECISION_TABLE “ShippingFeeCalculation” | |
| | INPUT | |
| | orderTotal: Number | |
| | destination: String | |
| | customerTier: String | |
| | OUTPUT | |
| | shippingFee: Number | |
| | deliveryTimeInDays: Number | |
| | RULES | |
| | \| orderTotal \| destination \| customerTier \| shippingFee \| | |
| | deliveryTimeInDays \| | |
| | \|------------\|-------------\|--------------\|-------------\|-------------------\| | |
| | \| >= 100 | \| “Domestic” \| * \| 0 \| 3 \| |
| | \| < 100 | \| “Domestic” \| “Premium” \| 4.99 \| 2 \| |
| | \| < 100 | \| “Domestic” \| “Regular” \| 7.99 \| 5 \| |
| | \| >= 200 | \| “International” \| * \| 15.00 \| 7 \| |
| | \| < 200 | \| “International” \| “Premium” \| 25.00 \| 5 \| |
| | \| < 200 | \| “International” \| “Regular” \| 35.00 \| 10 \| |

| | |
|---|---|
| | DESCRIPTION “Determine shipping fees and delivery times |
| | based on order details” |
| | END |
| | END |
| Fraud detection | RULESET “FraudDetection” VERSION “3.4” |
| rules | DESCRIPTION “Real-time transaction fraud detection rules” |
| | EXECUTION_STRATEGY FIRST_MATCH |
| | DATA_SOURCE “TransactionHistory” |
| | TYPE REST |
| | CONNECTION “https://api.financial-services.com/transactions” |
| | CACHE_DURATION 1 HOUR |
| | RETRY_POLICY 3 ATTEMPTS |
| | END |
| | RULE “UnusualLocation” |
| | WHEN |
| | NOT transaction.location WITHIN customer.frequentLocations |
| | AND transaction.amount > 1000 AND |
| | timeElapsedSince(customer.lastTransaction) < 2 HOURS |
| | THEN |
| | SET transaction.fraudScore += 75 |
| | SET transaction.flags.add(“UNUSUAL_LOCATION”) |
| | PRIORITY 200 |
| | AUDIT LEVEL DETAILED |
| | TRACE TRUE |
| | DESCRIPTION “Flag transactions from unusual locations” |
| | END |
| | RULE “RapidSmallTransactions” |
| | WHEN |
| | count(customer.transactions[t -> |
| | t.timestamp WITHIN LAST 1 HOUR AND |
| | t.amount < 50 |
| | ]) > 5 |
| | THEN |
| | SET customer.riskFlag = true |
| | CALL sendAlert(“RAPID_SMALL_TRANSACTIONS”, |
| | customer.id) |
| | PRIORITY 180 |
| | DESCRIPTION “Detect potential card testing patterns” |
| | END |
| | RULE “HighValueNewMerchant” |
| | WHEN |
| | transaction.amount > 2000 AND |
| | NOT transaction.merchantId IN customer.previousMerchants |
| | AND customer.accountAgeInDays < 30 |
| | THEN |
| | SET transaction.requiresVerification = true |
| | CALL initiateVerificationProcess(transaction, customer) |
| | PRIORITY 190 |
| | DESCRIPTION “Require verification for high-value purchases |
| | from new merchants by new customers” |
| | END |
| | RULE “PotentialAccountTakeover” |
| | WHEN |
| | transaction.deviceFingerprint != customer.knownDevices AND |
| | transaction.ipCountry != customer.homeCountry AND |
| | transaction.amount > 500 |
| | THEN |
| | SET transaction.status = “HELD” |
| | CALL sendMultiFactorAuthentication(customer) |
| | PRIORITY 210 |
| | DESCRIPTION “Hold suspicious transactions that indicate |
| | potential account takeover” |
| | END |
| | END |
| Insurance claims | RULESET “ClaimsProcessing” |
| processing rules | DESCRIPTION “Automated claims processing and routing rules” |
| | RULE “AutoApproval” |
| | WHEN |
| | claim.amount < 500 AND |
| | claim.type IN [“medical”, “dental”] AND |
| | policyholder.yearsActive > 2 AND |
| | count(policyholder.claims[c -> |
| | c.status == “approved” AND |
| | c.submissionDate WITHIN LAST 1 YEAR |
| | ]) < 3 |
| | THEN |
| | SET claim.status = “approved” |
| | SET claim.approvalPath = “auto” |
| | CALL initiatePayment(claim) |
| | PRIORITY 100 |
| | DESCRIPTION “Auto-approve low-risk small claims from |
| | established customers” |
| | END |
| | RULE “SpecialistRouting” |
| | WHEN |
| | claim.type == “property” AND |
| | claim.amount > 10000 |
| | THEN |
| | SET claim.assignedDepartment = “HighValueProperty” |
| | SET claim.reviewerLevel = “Senior” |
| | CALL notifySpecialistTeam(claim) |
| | PRIORITY 80 |
| | DESCRIPTION “Route high-value property claims to specialists” |
| | END |
| | RULE “FraudReview” |
| | WHEN |
| | ANY OF ( |
| | claim.submissionTime BETWEEN “22:00” AND “05:00”, |
| | claim.ipAddress IN suspiciousIpList, |
| | claim.documentCount < requiredDocuments[claim.type], |
| | claim.amount > 3 * average(policyholder.claims.amount) |
| | ) |
| | THEN |
| | SET claim.flaggedForReview = true |
| | SET claim.reviewReason = “Potential fraud indicators” |
| | EXECUTE_RULESET “FraudInvestigation” |
| | PRIORITY 150 |
| | AUDIT LEVEL DETAILED |
| | DESCRIPTION “Flag claims with potential fraud indicators for |
| | review” |
| | END |
| | END |

### Directed Graph Example

Real-world examples of decision-based directed graphs include (i) Medical diagnosis trees: starting with symptoms, moving through test results, leading to potential diagnoses and treatments; (ii) Customer support troubleshooting: beginning with the initial problem, following through various diagnostic questions, ending with specific solutions or escalation paths; (iii) Emergency response protocols: starting with incident type, moving through severity assessment, leading to specific response procedures; and (iv) Investment decision trees: beginning with risk assessment, evaluating market conditions, resulting in specific investment strategies.

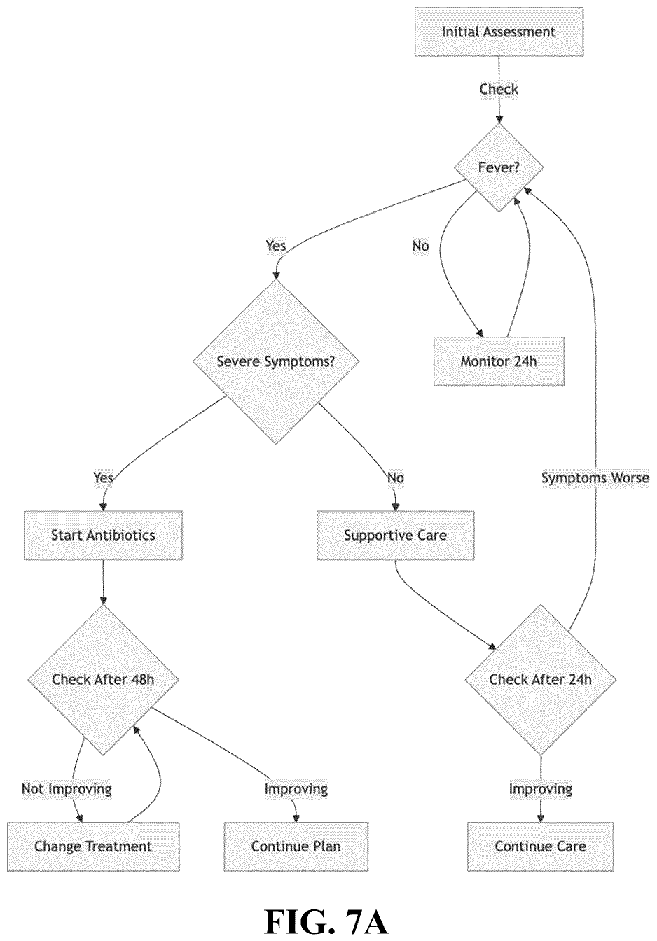

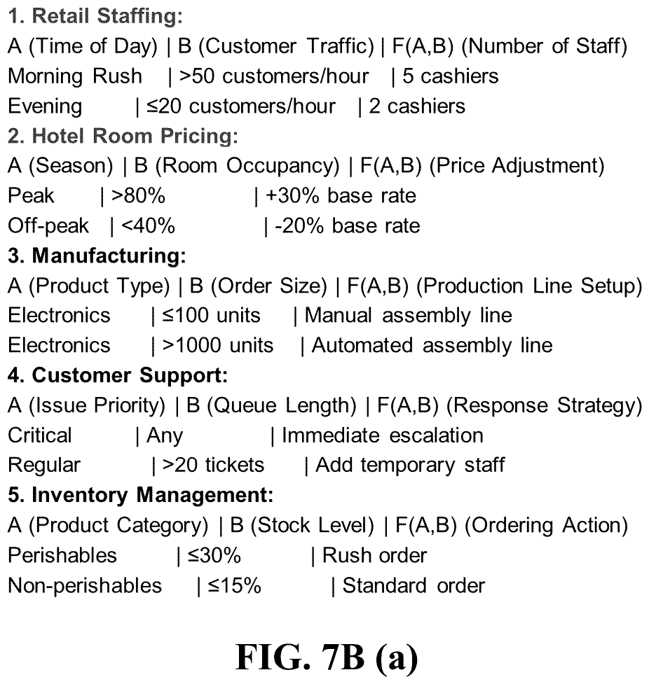

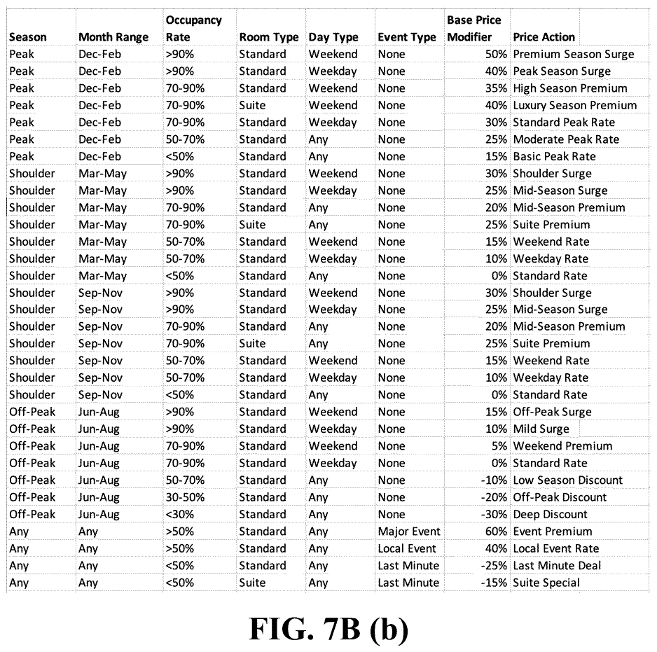

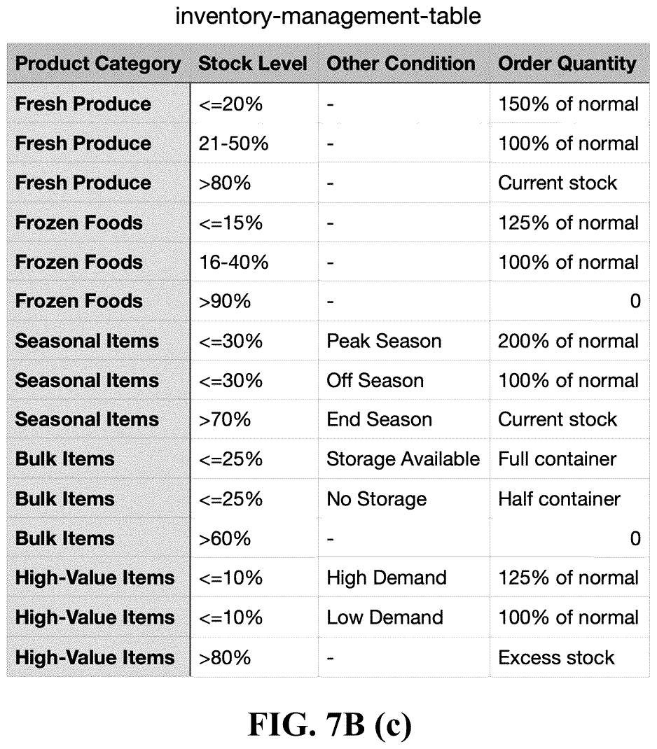

FIG. 7A shows the directed graph representing a medical diagnosis.

### Table Lookup Example

FIG. 7B, subpanels (a)-(c) each shows the table lookup applications for the exemplary system.

Integration. In the exemplary system, tasks can be partially or fully automated through flexible integration, particularly for external system integration. The exemplary system can employ interface management that present relevant tasks across multiple channels based on context. To this end, the exemplary system is not hardcoded and requirement development when integrating with external systems. In addition, the exemplary system is not confined to specific pre-defined interfaces.

Resilience. In the exemplary system, resilience is based on the transaction data state. No intermediate queues are needed. The exemplary system can recover based on transaction data and involves automatic next-step derivation. The exemplary system can make direct data element modification for corrections and automatic recalculation. The exemplary system can maintain only transaction data states. No workflow tracking is needed. The exemplary system can make direct changes to data elements and automatic task sequence adaptation. The exemplary system can perform a restart from the current transaction states. Intervention involves direct data element value modifications.

The exemplary system does not need to depend on workflow state queues and step tracking (improved system resilience). The exemplary system does not need complex recovery requiring queue and state management (improved recovery process). The exemplary system does not need to handle queues, clear steps, and redirect workflow (improved fault handling). The exemplary system does not need to track workflow states completion and queues (improved state management). The exemplary system does not need to employ process of reversing steps and clearing queues (improved error correction). The exemplary system does not need to perform a fresh and complete restart of the workflow (improved system restart). The exemplar system does not require a multiple-step process to modify workflow (improved intervention method).

User interaction. The exemplary system can coordinate dynamic task allocation across user groups based on data needs. The exemplary system can track all activities contributing to goal computation. In the exemplary system, users can receive dynamically assigned tasks based on the system state. Process adaptation can involve continuous updates based on data dependency. The exemplary system can learn and adapt from data relationships and execution patterns.

The exemplary system does not require predefined user roles and fixed assignments (improved multi-user coordination). Activity tracking is limited to predefined checkpoints (improved activity tracking). In the exemplary system, users do not need follow fixed sequences of steps (improved user interaction model). The exemplary system does not require manual process redesign (improved process adaptation). The exemplary system can learn as they follow programmed rules (improved system intelligence).

Example Decision Tree Created with CART (Classification and Regression Trees) in Machine Learning

An example decision tree created with CART (Classification and Regression Trees) is provided that could be used to predict, e.g., whether to approve a loan based on income and credit history, where the root node might ask “Is the applicant's credit history score high?” and then split into branches based on the answer, further asking questions like “Is the income high over $200K?” or “Is the income below $200K” to finally reach a leaf node stating “Approve loan” or “Do not approve loan” depending on the answer path taken through the tree. [1], [2], [3], [4], [5].

The CART may employ binary splits, meaning each node splits into only two branches (yes/no) based on a single feature comparison [2], [6], [7]. CART algorithm chooses the feature that best separates the data at each node, like “weather” in this case [2], [3], [6]. CART may be used for recursive splitting in which the process repeats on each child node until a stopping criteria is met (e.g., reaching a certain level of purity in the data) [2], [5], [6].

For CART, overfitting and regression tasks should be considered. Decision trees can easily overfit to training data, so techniques like pruning (removing unnecessary branches) are often used [5], [7], [8]. CART can also be used for regression problems, where the target variable is continuous (e.g., predicting house price based on features like square footage) [1], [9], [10].

Decision Tree Created with CART (Classification and Regression Trees) in Machine Learning Decision tree learning [17] is a supervised learning approach used in statistics, data mining, and machine learning. In this formalism, a classification or regression decision tree is used as a predictive model to draw conclusions about a set of observations.

Tree models where the target variable can take a discrete set of values are called classification trees; in these tree structures, leaves represent class labels, and branches represent conjunctions of features that lead to those class labels. Decision trees where the target variable can take continuous values (typically real numbers) are called regression trees. More generally, the concept of regression trees can be extended to any kind of object equipped with pairwise dissimilarities, such as categorical sequences [11]. Decision trees are among the most popular machine learning algorithms, given their intelligibility and simplicity [12].

In decision analysis, a decision tree can be used to visually and explicitly represent decisions and decision making. In data mining, a decision tree describes data (but the resulting classification tree can be an input for decision-making).

Decision tree learning is a method commonly used in data mining. [3] The goal is to create a model that predicts the value of a target variable based on several input variables.

A decision tree is a simple representation for classifying examples. For this section, assume that all of the input features have finite discrete domains and there is a single target feature called the “classification.” Each element of the domain of the classification is called a class. A decision tree or a classification tree is a tree in which each internal (non-leaf) node is labeled with an input feature. The arcs coming from a node labeled with an input feature are labeled with each of the possible values of the target feature, or the arc leads to a subordinate decision node on a different input feature. Each leaf of the tree is labeled with a class or a probability distribution over the classes, signifying that the data set has been classified by the tree into either a specific class or into a particular probability distribution (which, if the decision tree is well-constructed, is skewed towards certain subsets of classes).

A tree is built by splitting the source set, constituting the root node of the tree, into subsets-which constitute the successor children. The splitting is based on a set of splitting rules based on classification features [14]. This process is repeated on each derived subset in a recursive manner called recursive partitioning. The recursion is completed when the subset at a node has all the same values of the target variable, or when splitting no longer adds value to the predictions. This process of top-down induction of decision trees (TDIDT)[15] is an example of a greedy algorithm, and it is by far the most common strategy for learning decision trees from data [16].

In data mining, decision trees can also be described as the combination of mathematical and computational techniques to aid the description, categorization, and generalization of a given set of data.

A tree showing the approval of loans (“sibsp” is the number of spouses or siblings of the applicant). The figures under the leaves show the probability of having the loan approved and the percentage of observations in the leaf. Summarizing: Your chances of having the loan approve is good if you have (i) a high income or (ii) a not as high income and have a sufficient credit score.

### CONCLUSION

Computer-executable instructions, such as program modules, being executed by a computer may be used. Generally, program modules include routines, programs, objects, components, data structures, etc., that perform particular tasks or implement particular abstract data types. In its most basic configuration, the controller includes at least one processing unit and memory. Depending on the exact configuration and type of computing device, memory may be volatile (such as random-access memory (RAM)), non-volatile (such as read-only memory (ROM), flash memory, etc.), or some combination of the two. The controller may have additional features/functionality.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Machine Learning. In addition to the deep neural network described herein, the analysis system can be implemented using one or more artificial intelligence and machine learning operations. The term “artificial intelligence” can include any technique that enables one or more computing devices or comping systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target) during training with a labeled data set (or dataset). In an unsupervised learning model, the algorithm discovers patterns among data. In a semi-supervised model, the model learns a function that maps an input (also known as a feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers, such as an input layer, an output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similarly to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifiers performance (e.g., an error such as L1 or L2 loss), during training. This disclosure contemplates that any algorithm that finds the minimum of the cost function can be used. LR classifiers are known in the art and are therefore not described in further detail herein.

An Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier's performance during training. This disclosure contemplates any algorithm that finds the maximum or minimum. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

Although exemplary implementations may refer to utilizing aspects of the presently disclosed subject matter in the context of one or more stand-alone computer systems, the subject matter is not so limited but rather may be implemented in connection with any computing environment, such as a network or distributed computing environment. Still further, aspects of the presently disclosed subject matter may be implemented in or across a plurality of processing chips or devices, and storage may similarly be implemented across a plurality of devices. Such devices might include personal computers, network servers, handheld devices, and wearable devices, for example.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications and publications as listed below and throughout this document are hereby incorporated by reference in their entirety herein.

### REFERENCES

[1] https://www.coursera.org/articles/decision-tree-machine-learning
[2] https://www.youtube.com/watch?v=DCZ3tsQIoGU
[3] https://www.sthda.com/english/articles/35-statistical-machine-learning-essentials/141-cart-model-decision-tree-essentials/
[4] https://www.youtube.com/watch?v=s5Plqr0Y7D8
[5] https://medium.com/geekculture/decision-trees-with-cart-algorithm-7e179acee8ff
[6] https://www.geeksforgeeks.org/implementing-cart-classification-and-regression-tree-in-python/
[7] https://medium.com/@akashgopalgs/cart-vs-id3-unraveling-the-secrets-of-decision-tree-algorithms-de8feldacc52
[8] https://koalaverse.github.io/machine-learning-in-R/decision-trees.html
[9] https://medium.com/@kaanerdenn/decision-trees-in-machine-learning-cart-and-advanced-trees-8b3fe375e9f9
[10] https://www.geeksforgeeks.org/cart-classification-and-regression-tree-in-machine-learning/
[11] Studer, Matthias; Ritschard, Gilbert; Gabadinho, Alexis; Müller, Nicolas S. (2011). “Discrepancy Analysis of State Sequences”. Sociological Methods & Research. 40 (3): 471-510. doi: 10.1177/0049124111415372. ISSN 0049-1241. S2CID 13307797.
[12] Wu, Xindong; Kumar, Vipin; Ross Quinlan, J.; Ghosh, Joydeep; Yang, Qiang; Motoda, Hiroshi; McLachlan, Geoffrey J.; Ng, Angus; Liu, Bing; Yu, Philip S.; Zhou, Zhi-Hua (2008-01-01). “Top 10 algorithms in data mining”. Knowledge and Information Systems. 14 (1):1-37. doi:10.1007/s10115-007-0114-2.hdl:10983/15329. ISSN 0219-3116. S2CID 2367747.
[13] Rokach, Lior; Maimon, O. (2014). Data mining with decision trees: theory and applications, 2nd Edition. World Scientific Pub Co Inc. doi: 10.1142/9097. ISBN 978-9814590075. S2CID 44697571.
[14] Shalev-Shwartz, Shai; Ben-David, Shai (2014). “18. Decision Trees”. Understanding Machine Learning. Cambridge University Press.
[15] Quinlan, J. R. (1986). “Induction of decision trees” (PDF). Machine Learning. 1:81-106. doi: 10.1007/BF00116251. S2CID 189902138.
[16] Rokach, L.; Maimon, O. (2005). “Top-down induction of decision trees classifiers-a survey”. IEEE Transactions on Systems, Man, and Cybernetics-Part C: Applications and Reviews. 35 (4): 476-487. CiteSeerX 10.1.1.458.7031.doi:10.1109/TSMCC.2004.843247. S2CID 14808716.
[17] Source: https://en.wikipedia.org/wiki/Decision_tree_learning

What is claimed:

1. A system comprising:
a task management engine configured to (i) create a template of data elements for a new transaction having a decision-making operation, (ii) create an initial task pool for the transaction, and (iii) monitor changes in a data element and data dependencies within the transaction,
wherein the decision-making operation is performed via an updatable data structure comprising a data computation method per the task management engine,
wherein the transaction comprises a container or instance that holds a set or template of data elements,
wherein the data element defines a piece of information that is read and updatable by a task or computed by a data computation operation that defines how data elements are derived via a set of logics contained in the updatable data structure,
wherein the data computation operation implicitly creates data dependencies among data elements that are dynamically updatable for the transaction,
wherein at runtime, each transaction data element and data computation method form a dynamic data dependency,
wherein a change to one data element causes (i) re-computation of other data elements that depend on the respective data element and (ii) initiate or close tasks; and
a task pool operatively coupled to the task management engine, wherein the task pool comprises a logical container of all pending or in-progress tasks to be performed for all active transactions, wherein a new task is created and added to the task pool when the task management engine detects a data element requiring an update.

2. The system of claim 1, wherein the task management engine is configured to:
analyze the transaction for incomplete information in data elements,
analyze the transaction for new data elements needing verification,
automatically create or update tasks in the task pool to respectively collect required information,
assign a task that performs a direct manual operation, or
assign a task that performs a computerized operation.

3. The system of claim 1 comprising:
an interface configured to curate a user portal to receive input from a user to inputs/edits data, wherein the interface is configured to retrieve a task from the task pool and complete an operation.

4. The system of claim 1, wherein the task management engine is configured to update, in a database, a status field of a data element to an edited status upon the data element being edited, and wherein the task management engine is configured to (i) re-compute other data elements that depend on the respective data element and (ii) initiate or close tasks.

5. The system of claim 1, wherein the task management engine is configured to update, in a database, a status field of a data element to a verified status upon the data element being verified.

6. The system of claim 1, wherein the updatable data structure is iteratively created, via an editor, from input from individuals in an organization servicing the transaction.

7. The system of claim 1, wherein the updatable data structure is generated by a machine learning operation using training data and existing data structure from the organization.

8. The system of claim 7, wherein the machine learning operation comprises a classification and regression tree configured to perform a combination of: (i) empirically define cut-offs for branching or splitting of numerical data elements, and (ii) empirically define placements of data elements within the updatable data structure,
(iii) determine new branches responsive to new data received in relation to the training data, and
(iv) prune unnecessary branches within the updatable data structure.

9. The system of claim 1, wherein the transaction is directed to a loan or insurance application, and wherein the decision operation is directed to an approval, declination, or classification of the loan or insurance application within a pipeline operation of the task management engine.

10. The system of claim 1, wherein the transaction is directed to a building permit application or research grant proposal, and wherein the decision operation is directed to an approval, declination, or request for more additional for the building permit application or the research grant proposal using a pipeline operation of the task management engine.

11. The system of claim 1, wherein the transaction is directed to a patient check-in process for a clinic, hospital, or healthcare service facility, and wherein the decision operation is directed to a routing operation for the patient check-in process using a pipeline operation of the task management engine.

12. The system of claim 1, wherein the transaction is directed to a collateral of a collateralized financial product, wherein the decision operation is directed to validation or maintenance of the collateralized financial product using a pipeline operation of the task management engine.

13. The system of claim 1, wherein the transaction is directed to an insurance claim application, and wherein the decision operation is directed to an approval, declination, or request for additional information for the insurance claim application using a pipeline operation of the task management engine.

14. The system of claim 1, wherein decision outcomes in the data computation methods are defined by one or multiple data elements.

15. The system of claim 1, wherein the data computation method is one of the following: (i) a directed acyclic graph, (ii) a directed cyclic graph,
(iii) a weighted directed graph, (vi) a decision tree, (v) a set of rules, (vi) a formula, and (vii) a table.

16. The system of claim 1 further comprising a path analyzer analyzing all possible paths from a decision outcome to a root node.

17. The system of claim 16, wherein the path analyzer identifies and evaluates multiple paths for efficiency, priority, and conflict resolution.

18. The system of claim 16, wherein the path analyzer identifies and evaluates a loop in a path.

19. A computerized method comprising:
providing a task management engine configured to (i) create a template of data elements for a new transaction requiring a decision-making operation, (ii) create an initial task pool for the transaction, and (iii) monitor changes in a data element and data dependencies within the transaction;
analyzing the transaction for incomplete information in data elements;
analyzing the transaction for new data elements needing verification;
automatically creating or updating tasks in the task pool to respectively collect required information;
assigning a task that performs a direct manual operation; and
assigning a task that performs a computerized operation.

20. A non-transitory computer-readable medium having instructions stored thereon, wherein execution by a processor causes the processor to:
analyzing a transaction for incomplete information in data elements;
analyzing the transaction for new data elements needing verification;
automatically creating or updating tasks in a task pool to respectively collect required information;
assigning a task that performs a direct manual operation; and
assigning a task that performs a computerized operation.
